(19) United States
(12) Patent Application Publication
(10) Pub. No.: US 2026/0027218 A1
(43) Pub. Date: Jan. 29, 2026

(54) STAR-SHAPED GRAFT COPOLYMERS

(71) Applicant: POLYPEPTIDE THERAPEUTIC SOLUTIONS, S.L., PATERNA (ES)

(72) Inventors: Rafael MIRAVET MARTÍ, PATERNA (ES); Luz TORTAJADA, PATERNA (ES); Lidia HERRERA MUÑOZ, PATERNA (ES); Raquel ROMAN, PATERNA (ES); Juan José ARROYO CRESPO, PATERNA (ES); Carles FELIP LEON, PATERNA (ES); Maria Jesús VICENT DOCÓN, VALENCIA (ES); Vicent Josep NEBOT CARDA, PATERNA (ES)

(21) Appl. No.: 18/867,392

(22) PCT Filed: May 25, 2023
(86) PCT No.: PCT/EP2023/064072

§ 371 Date: Nov. 19, 2024

(30) Foreign Application Priority Data
May 26, 2022 (EP) 22382505.0

(51) Int. Cl. A61K 47/59; C08G 83/00

(52) U.S. Cl. CPC A61K 47/595; C08G 83/003

(57) ABSTRACT

New star-shaped polymers comprising graft copolymer backbone arms, which e.g. are useful for delivering active agents and/or imaging agents to target cells or tissues.

### FIELD OF THE INVENTION

The present invention relates to new star-shaped polymers comprising graft copolymer backbone arms, which e.g. are useful for delivering active agents and/or imaging agents to target cells or tissues.

### BACKGROUND

There has been a considerable effort to the development of new polymeric structures with specific properties to be used as targeted drugs, including large molecules such as polypeptides and nucleic acids, delivery systems.

Delivery of active ingredients to a target site located inside the cells requires appropriate delivery carriers, which provide adequate protection and permit the active ingredients to be efficiently delivered to specific tissues within the body.

The delivery carrier must overcome different extracellular and intracellular barriers to reach their target sites inside the cell. Although viral vectors may be more efficient for gene delivery compared to non-viral vectors, their use is accompanied by several risks including toxicity, immunogenicity, and limitations in size of the genetic material cargo. Non-viral vectors are generally safer and more convenient for large scale production.

EP3331937B1 discloses non-viral vectors made from a family of 3-arm star shaped polypeptide derivatives consisting of a 1,3,5-benzenetricarboxamide related central core employed as the initiator for the ring opening polymerization of N-carboxyanhydride monomers and 3 polypeptide backbone arms. The novel graft copolymers compounds described herein are not directly and unambiguously disclosed in EP3331937B1.

### SUMMARY OF THE INVENTION

The problem to be solved by the present invention may be seen as to provide new graft copolymers with novel/improved properties and which are able to deliver e.g. active agents and/or imaging agents to target cells or tissues.

According to art—the term “graft copolymer” relates to a general class of segmented copolymers and generally consist of a linear backbone of one composition and randomly distributed branches of different composition (see e.g. FIG. 1 herein for an illustration).

As discussed above—EP3331937B1 discloses non-viral vectors made from a family of 3-arm star shaped polypeptide derivatives. As known in the art-Star polypeptides are branched polymers, which consist of various linear chains linked to a central core.

Claim 4 of EP3331937B1 discloses a compound structure, covering e.g. a “R₂ . . . ” Lysine (Lys) or Glutamic acid (Glu) side chain radical bound to a so-called CL radical, which is a broadly defined (C₁-C₅₀₀)-alkyl that may be substituted at different places. The CL radical does not directly and unambiguously describe a herein relevant amino acid structure—i.e. this document does not directly and unambiguously describe a structure, wherein a “R₂ . . . ” amino acid side chain of the R arm structure of claim 4 is bound to an amino acid based structure.

Said in other words, EP3331937B1 does not disclose a graft copolymer, wherein amino acid side chains of the linear backbone are bound to branches with an amino acid structure.

Consequently, and just as illustrative examples—EP3331937B1 does not disclose a graft copolymer structure, wherein R₂ . . . is Lys (PLys) side chain and it is bound to e.g. a glutamic acid (PGA) based branch structure (see e.g. illustrative example “StPLys-g-PGA” structure of FIG. 2 herein) or R₂ . . . is Glu (PGA) side chain and it is bound to sarcosine (PSar) based branch structures (see e.g. illustrative example “PGA-g-PSar-g-PHis” structure of FIG. 2 herein).

Overall and without being limited to theory-one may say that the present invention is based on that the present inventors identified that graft copolymers with novel/improved properties over the Star polypeptide structures in EP3331937B1 may be obtained by making graft copolymers, wherein e.g. Lys/Orn (Ornithine) and/or Glu/Asp (Aspartic acid) amino acid side chains of the linear backbone are bound to branches with an amino acid structure.

The novel graft copolymers discussed herein may overall herein be termed Starbrushes.

As discussed in further detail below (see e.g. working Examples), the novel graft copolymers discussed herein may have a number of different useful advantages—such as e.g. a molecular weight (MW) that is so high that a self-assembly process may not be required in order to make structures in the nanometer range (e.g. 4-300 nm in radius) that can be useful for delivery of e.g. active drug agents to target cells.

As discussed in e.g. of EP3331937B1, the Star polypeptides discussed therein generally need to undergo a self-assembly process to make structures in the nanometer range, something that on industrial relevant scale may be complicated to control—accordingly, the fact that self-assembly process may not be required for the novel Starbrush graft copolymers discussed herein may be seen as an advantage over Star polypeptides described in EP3331937B1.

A further advantage of the novel graft copolymers discussed herein relates to that by choosing different/suitable amino acid side chains with characteristics of interest (e.g. hydrophobic/hydrophilic or cationic/anionic characteristics)-one has a huge flexibility to make graft copolymer compounds for a required purpose of interest.

For instance—Example 1 below describes examples of graft copolymers designed to obtain unimolecular aggregates with hydrophobic pockets with the capability of encapsulating hydrophobic payloads/agents of interest-(see e.g. compounds 8-11 below).

One may e.g. also make graft copolymers with different degrees of both hydrophobic and hydrophilic properties (see e.g. compounds 8-12 of Example 1 below).

One may e.g. also conjugate an agent of interest (e.g. a cell-targeting agent such as e.g. a small molecule or e.g. a therapeutic peptide) to a graft copolymer as described herein—see e.g. example compounds 15, 18, 22 discussed below.

Accordingly, a first aspect of the invention relates to graft copolymer, wherein the graft copolymer is a compound of formula Ia, a pharmaceutically acceptable salt thereof, or any stereoisomer or mixtures of stereoisomers, either of the compound of formula (Ia) or of any of its pharmaceutically acceptable salts, comprising homo-polypeptides or random or block or graft co-polypeptides:

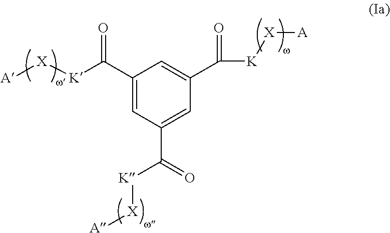

wherein A, A′ and A″ are each independently selected from a polymer comprising a backbone of repeating structural units of formula (I); and each of A, A′ and A″ sub-units may be same or different;
wherein K, K′ and K″ are each independently selected from —O—, —NH—;
X is selected from

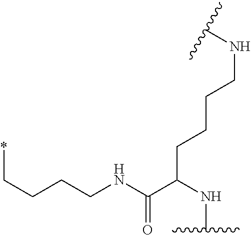 [SMILES: *CCCCNC(=O)C(CCCCN*)N*]

wherein ω, ω′ and ω″ are an integer from 0 to 1; each wavy lines denote the attaching points to A, A′ or A″; and “*” denotes the attaching point to K, K′ or K″;
and wherein formula (I) is a graft copolymer compound comprising a backbone of repeating structural units of formula (I)

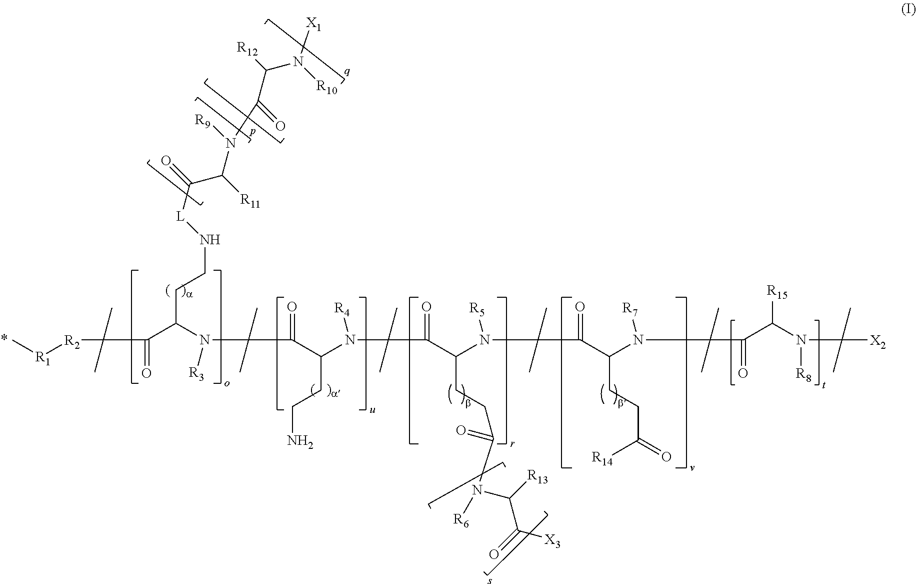

wherein though the main repeating units defined by square brackets with their numerical value o, r, t, u, and v, respectively, are shown in a particular order for convenience of description, the main repeating units may be present in any order and may be block or randomly present;
wherein each of the main repeating unit defined by square brackets with the numerical value “o” may be same or different between each other, may comprise blocks of secondary monomer units defined by square brackets with the numerical value “p” and “q” which may be same or different from each other according to the definitions of its different substituents;
wherein each of the main repeating units defined by square brackets with the numerical value “r” may be same or different between each other, may comprise blocks of secondary monomer units defined by square brackets with the numerical value “s” and “t” which may be same or different from each other according to the definitions of its different substituents;
wherein the “*” denotes an attaching point;
wherein “/” indicates that the sequential order of the repeating units on both sides of the sign are arbitrary;
R₁ is a biradical selected from the group consisting of

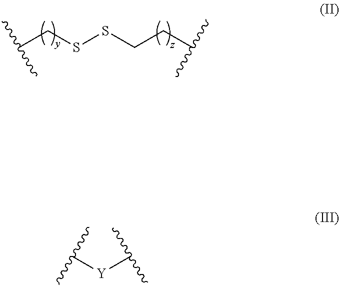

wherein the wavy lines denote the attaching points;
wherein y and z are integers independently ranging from 1 to 20; and
wherein Y is a biradical with a molecular weight (MW) from 5 to 3000 g/mol
R₂ is selected from —O—, —NH—;
wherein o and r are an integer each independently selected from 0 to 5000; with the proviso that at least one of o or r is ≠0;
p and s are an integer each independently selected from 1 to 1000;
q, t, u and v are an integer each independently selected from 0 to 1000;
α, and α′ are each independently an integer selected from 1 to 4;
β, and β′ are each independently an integer selected from 0 to 3;
each R₃ is independently selected from H and —CH₃;
each R₄ is independently selected from H and —CH₃;
each R₅ is independently selected from H and —CH₃;
each R₆ is independently selected from H and —CH₃;
each R₇ is independently selected from H and —CH₃;
each R₈ is independently selected from H and —CH₃;
each R₉ is independently selected from H and —CH₃;
each R₁₀ is independently selected from H and —CH₃;
each R₁₅ is independently selected from H and —CH₃;
each R₁₁ and R₁₂ is independently selected from H, an imaging or labeling agent, a cell-targeting agent, and a radical of formula (XII), (XIII), (XIV), (XV), (XVI), (XVII), (XVIII), (XIX), (XX), (XXI), (XXII) or (XXIII)

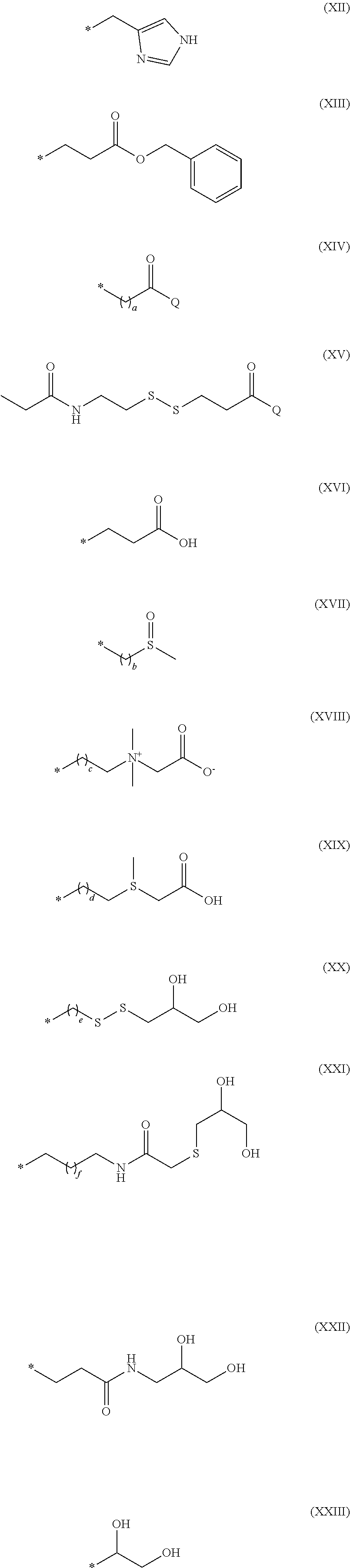

wherein Q is selected from —OH, an imaging or labeling agent, a cell-targeting agent, and
a, b, c, d, e and f are integers each independently selected from 0 to 4;
each R₁₃ is independently selected from H, —CH₃, an imaging or labeling agent, a cell-targeting agent, and a radical of formula (XII), (XIII), (XIX), (XX), (XXI), (XXII), (XVIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), or (XXXVIII)

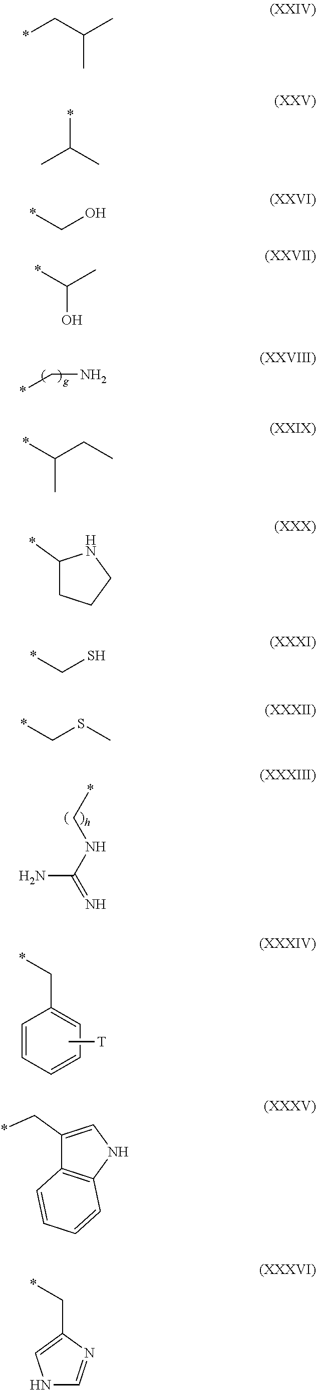

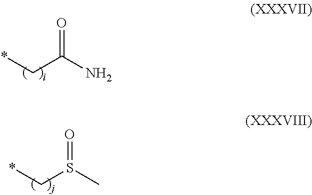

wherein g, h, i and j are integers each independently selected from 0 to 4;
T is selected from H, and —OH;
each R₁₄ is independently selected from H, —OH, an imaging or labeling agent, and a cell-targeting agent;
wherein L is selected from a single bond, and a biradical of formula (XXXIX), (XL), (XLI), (XLII), (XLIII), or (XLIV)

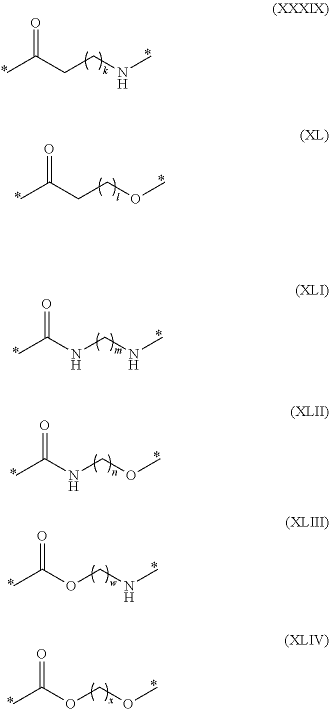

wherein k, l, m, n, w, and x are integers each independently selected from 0 to 4;
wherein the “*” denotes the attaching points;
each X₁ is independently selected from H, —C(O)H, —S(O)H, —NHCH(CH₃)₂, —(C₁-C₄)alkylNH₂, —(C₁-C₄)alkylNHCH₃, —(C₁-C₄)alkylN(CH₃)₂, —O—(C₁-C₄)alkyl-NH₂, —O—(C₁-C₄)alkyl-NHCH₃, —O—(C₁-C₄)alkyl-N(CH₃)₂, an imaging or labeling agent, a cell-targeting agent, and a radical of formula (XVI), (XVII), (XVIII), (XIX), (XX), (XXI), (XXII) or (XXIII);
each X₂ is independently selected from H, —C(O)H, —S(O)H, —NHCH(CH₃)₂, —(C₁-C₄)alkylNH₂, —(C₁-C₄)alkylNHCH₃, —(C₁-C₄)alkylN(CH₃)₂, —O—(C₁-C₄)alkyl-NH₂, —O—(C₁-C₄)alkyl-NHCH₃, —O—(C₁-C₄)alkyl-N(CH₃)₂, an imaging or labeling agent, a cell-targeting agent, and a radical of formula (XVI), (XVII), (XVIII), (XIX), (XX), (XXI), (XXII) or (XXIII); and
each X₃ is independently selected from H, —C(O)H, —S(O)H, —NHCH(CH₃)₂, —(C₁-C₄)alkylNH₂, —(C₁-C₄)alkylNHCH₃, —(C₁-C₄)alkylN(CH₃)₂, —O—(C₁-C₄)alkyl-NH₂, —O—(C₁-C₄)alkyl-NHCH₃, —O—(C₁-C₄)alkyl-N(CH₃)₂, an imaging or labeling agent, a cell-targeting agent, and a radical of formula (XVI), (XVII), (XVIII), (XIX), (XX), (XXI), (XXII) or (XXIII).

Just for illustration in relation to the scope of the first aspect are below discussed illustrative example structures of Figures herein.

The example “St-PLys-g-PGA” structure (alternatively termed St-PLys-g-PGlu”) of FIG. 2 herein may in the language of the first aspect be seen as a structure, wherein grafted Lys/Orn related units are present (i.e. integer “o” is ≠0) and grafted Glu/Asp related units are not present (i.e. integer “r” is 0).

The term “St” in the “St-PLys-g-PGA” nomenclature relates to a central core structure within the scope of formula (Ia) of the first aspect-such as e.g. Star 1,3,5-benzenetricarboxamide ring related central core of the illustrated structure of FIG. 2.

The example “St-PLys-g-PHis-b-PGA” structure of FIG. 2 also comprises a PGA block (b)—i.e. integer “q” is ≠0.

The example “St-PGA-g-PSar-g-PHis” structure of FIG. 2 may in the language of the first aspect be seen as a structure, wherein grafted Glu/Asp related units are present (i.e. integer “r” is ≠0) and grafted Lys/Orn related units are not present (i.e. integer “o” is 0).

The example St-PLys(18)-g-PHis(5)-b-PSar(20) of compound 10D below—may in the language of the first aspect be seen as a structure, wherein integer “o” is 18, integer “p” is 5, and integer “q” is 20.

As discussed above—the first aspect reads “each of the main repeating units defined by square brackets with the numerical value “r” may be same or different between each other, may comprise blocks of secondary monomer units defined by square brackets with the numerical value “s” and “t” which may be same or different from each other according to the definitions of its different substituents”.

Accordingly, the illustrative “PGA-g-PSar-g-PHis” structure of FIG. 2 is an example of a situation within the scope of claim 1, wherein the “s” units are different since some comprises PSar and others comprises PHis substituents.

According to art and illustrated in FIG. 1 herein:
the term “Grafting from” relates to a method, wherein the linear backbone comprises active sites capable of initiating the polymerization, and therefore is a functional group that serves as an initiator to graft from it; and
term “Grafting to” relates to a method that involves the use of a backbone chain with functional groups that are not used to initiate the polymerization as such, and therefore may be said to be used as an “attachment point” for pre-synthesized polymers.

As known in the art—the “H₃N⁺” (amino) group of a Lys side-chain (see e.g. “St-PLys-g-PGA” structure of FIG. 2) may be a backbone initiator active site in a “Grafting from” method discussed herein—i.e. the amine group serves as an initiator to graft from it.

As known in the art—the “COO” (carboxyl) group of a Glu side-chain (see e.g. “PGA-g-PSar-g-PHis” structure of FIG. 2) may be a backbone “attachment point” functional group in a “Grafting to” method discussed herein—i.e. the carboxyl group is used as an “attachment point” for e.g. pre-synthesized polymers through their terminal amine.

In short—“Grafting from” and “Grafting to” are well known methods for the skilled person—i.e. the skilled person may, based on the technical information disclosed herein and common general knowledge routinely, identify a suitable process for the synthesis of a graft copolymer compound of the first aspect—i.e. the skilled person may routinely make compound of the first aspect.

In formula (I) of first aspect—main repeating unit defined by square brackets with the numerical value “t” may be seen as related to use of e.g. Alanine/Sarcosine/Glycine (Ala/Sar/Gly).

Several working examples herein describe structures where the integer “t” is zero—accordingly, it may be preferred that the integer “t” is 0.

As understood by the skilled person in the present context—the term “comprising” within the language of the first aspect reading “A graft copolymer compound comprising a backbone of repeating structural units of formula (I)” should be understood as a graft copolymer compound of the invention may comprise other structures (e.g. repeating units) than explicitly shown for the formula (I) of the first aspect.

For instance, a graft copolymer compound of the invention may comprise a repeating unit similar to unit “t”, wherein the unit comprises amino acid related structures that are different from Ala/Sar related amino acids of unit “t” (such as for instance Arginine(Arg), Cysteine(Cys), etc).

A second aspect of the invention relates to a polymer complex comprising a graft copolymer of the first aspect and/or embodiment thereof, and at least a pharmaceutical, veterinary or cosmetical active agent.

As discussed herein-based on common general knowledge and the technical disclosure herein—it is routine work for the skilled person to make a polymer complex of the second aspect and/or embodiment thereof of the present invention.

A third aspect of the invention relates to a pharmaceutical, veterinary or cosmetic composition comprising at least one polymer complex of the second aspect and/or embodiment thereof, together with one or more appropriate excipients or carriers.

Based on common general knowledge and the technical disclosure herein—it is routine work for the skilled person to make pharmaceutical, veterinary or cosmetic composition of the third aspect and/or embodiment thereof of the present invention.

A fourth aspect of the invention relates to a polymer complex of the second aspect and/or embodiment thereof, or the pharmaceutical composition according of the third aspect and/or embodiment thereof, for use as a medicament.

A fifth aspect of the invention relates to a polymer complex of the second aspect and/or embodiment thereof, or the pharmaceutical composition according of the third aspect and/or embodiment thereof, for use (i) as transfection reagent for transfecting at least one active agent into a cell; (ii) for use in the in vivo or ex vivo production of biologics encoding a recombinant protein, a peptide or an antibody, or in the production of recombinant virus; (iii) for use as a therapeutic or prophylactic vaccine against viral infections or as a therapeutic vaccine against cancers; or (v) for use in genome engineering, for cell reprogramming, for differentiating cells or for gene-editing.

Based on common general knowledge and the technical disclosure herein—it is routine work for the skilled person to identify a suitable medical use of a polymer complex and/or a pharmaceutical composition described herein.

For instance, many of the medical uses described in the art for related polymer compounds (see e.g. above discussed EP3331937B1) may also be relevant for the graft copolymer compounds of the present invention.

A sixth aspect of the invention relates to a method for delivering a pharmaceutical active agent (e.g. a nucleic acid) into a target cell, which comprises administering a composition (e.g. solution) that contains the polymer complex of the second aspect and/or embodiment thereof to an animal, including human, so that the complex is getting into physical contact with the target cell and thereby delivers the pharmaceutical active agent into the cell.

Based on common general knowledge and the technical disclosure herein—it is routine work for the skilled person to identify a suitable method to deliver an active agent of interest to a target cell of interest.

Working examples herein describe that graft copolymer compounds of the present invention may get into physical contact with a target cell of interest and thereby delivers an active agent of interest into the cell.

Further, related polymer compounds (see e.g. above discussed EP3331937B1) are also described as being useful for delivering an active agent into a target cell of interest.

A seventh aspect of the invention relates to a process for preparing a compound which is structurally different from a graft copolymer starting compound of the first aspect and/or embodiment thereof, comprising following steps:
(i): using a compound of the first aspect and/or embodiment thereof as a starting compound;
(ii) making structural changes to the compound of step (i) to obtain a compound, which is structurally different from the starting compound of (i).

Based on common general knowledge and the technical disclosure herein—it is routine work for the skilled person to use a graft copolymer compound as described herein as a starting compound to make a structurally different compound.

Embodiments of the present invention are described below, by way of examples only.

As evident to the skilled person in the present context—a herein described preferred embodiment (e.g. a preferred radical structure) may preferably be combined with another described preferred embodiment (e.g. another preferred radical structure)—i.e. a combination of two individual herein described preferred embodiments is understood to be a more preferred embodiment.

### DRAWINGS

FIG. 1: Illustrative example of grafting from/to methods for making a graft copolymer.

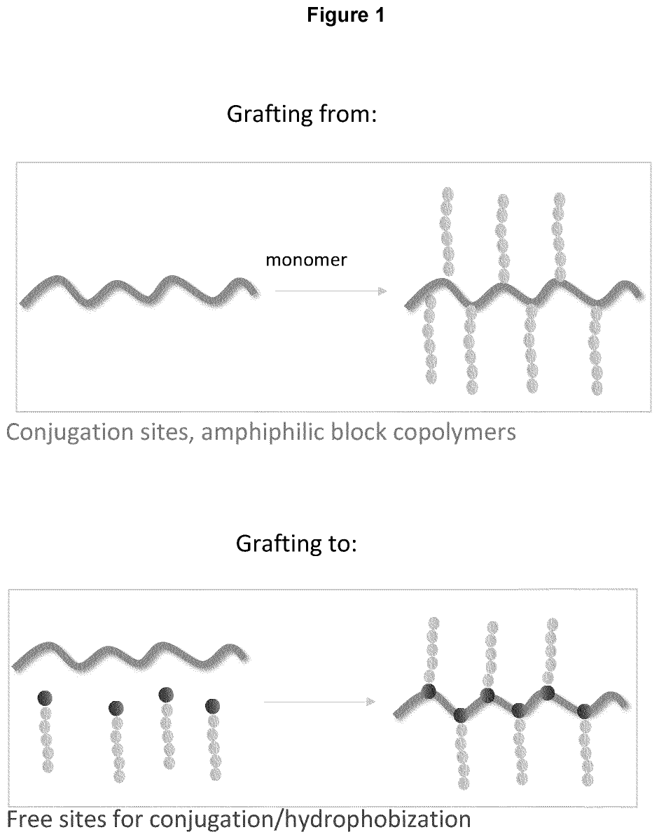

FIG. 2: Illustrative examples of graft copolymer compounds/structures within the scope of first aspect (i.e. claim 1) herein.

### DETAILED DESCRIPTION OF THE INVENTION

### Graft Copolymer of First Aspect—Star Core Structure

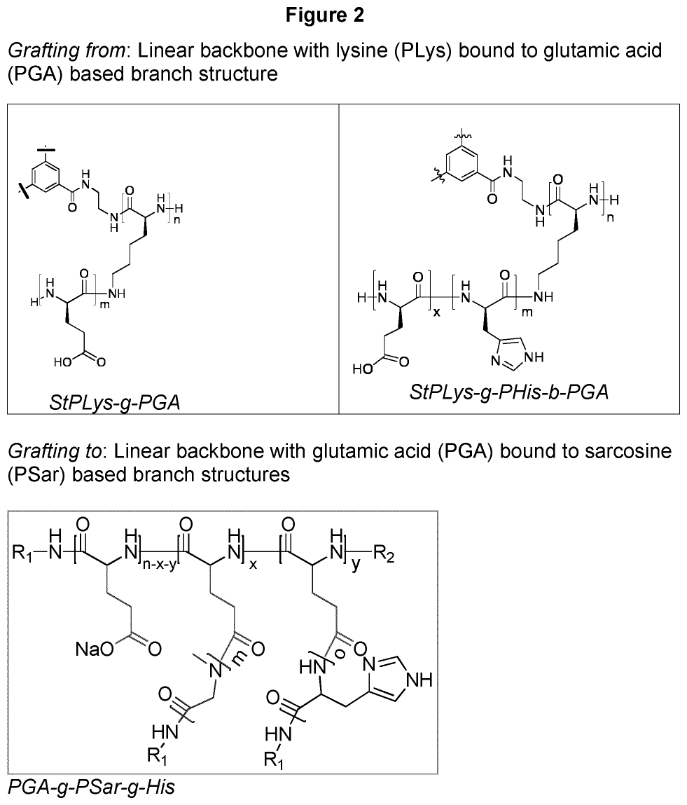

In a number of compounds of working examples herein (see e.g. FIG. 2 and working examples below)—the compounds comprise a Star related central core similar to the Star core described in above discussed EP3331937B1.

The structure X may be seen as providing the possibility of attaching even further branches to the Star core structure.

It may be preferred (as e.g. in graft copolymer compounds of working examples herein) that structure X is not present—i.e. that ω, ω′ and ω″ integer is 0.

Preferably K, K′ and K″ are each —NH—.

As discussed above and in working Examples herein, the novel graft copolymers discussed herein may have a molecular weight (MW) that is so high that a self-assembly process may not be required in order to make structures in the nanometer range (e.g. 4-300 nm in radius) that can be useful for delivery of e.g. active drug agents to target cells.

Accordingly, in a preferred embodiment the graft copolymer of the first aspect and herein relevant embodiments thereof is a graft copolymer, wherein the molecular weight (MW) is from 3 to 500000 kilodaltons (kDa)—more preferably from 10 to 100000 kilodaltons (kDa), even more preferably from 10 to 20000 kilodaltons (kDa), and most preferably from 20 to 10000 kilodaltons (kDa).

As known in the art—the molecular weight (MW) of a compound with a known structure may be calculated/determined via information from the periodic table.

For a suitable experimental MW determination method—see e.g. “Size Exclusion Chromatography (SEC)” analytical method of our Examples text herein.

A preferred embodiment relates to a graft copolymer of the first aspect and herein relevant embodiments thereof, wherein the graft copolymer is a unimolecular structure with a radius from 4-300 nm.

For a suitable radius determination method—see e.g. “Size Distribution” analytical method of our Examples text herein.

Graft Copolymer of First Aspect—Radicals R₁ and R₂

As discussed above—R₂ is selected from —O—, —NH—.

In several herein exemplified structures is R₂=—NH—

Accordingly, it is preferred that R₂=—NH—

As discussed above—R₁ is a biradical selected from the group consisting of

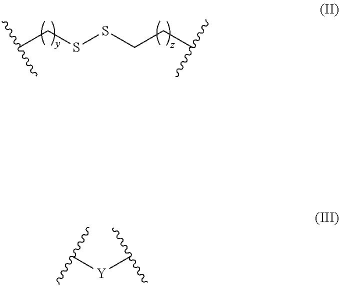

wherein the wavy lines denote the attaching points;
wherein y and z are integers independently ranging from 1 to 20; and
wherein Y is a biradical with a molecular weight (MW) from 5 to 3000 g/mol.

Radical R₁ may essentially be seen as a covalent linker radical—i.e. a linker to the left end “*” attaching point of formula (I) of the first aspect.

In the Star polypeptide illustrative structures of FIG. 2 herein is R₁=ethyl—i.e. the “R₁=ethyl” linker is used to attach/bind the shown Star related central core to the left end “*” attaching point of the shown graft copolymer of formula (I)—see also Star polypeptide formula (Ia) herein.

The R₁ linker related radical may be seen as corresponding to the similar R₁ linker related radical of e.g. claim 1 of above discussed EP3331937B1.

As discussed in this EPB1 patent (see e.g. claim 1 and workings Examples—e.g. Table 1 on page 29)—such R₁ linker related radicals may be many different suitable structures—for instance a structure comprising a in a human cell in vivo cleavable disulfide bond.

In short—the skilled person knows from the art numerous different suitable linker related structures of different molecular weight (MW) sizes.

It may be preferred that Y is a biradical with a molecular weight (MW) from 8 to 1000 g/mol or more preferably from 2 to 500 g/mol.

Preferably, the Y biradical of R₁ is a biradical selected from the group consisting of —NH—, —NH(C₁-C₆)alkyl-, —O—, —(C₁-C₆)alkyl-COO—, a straight or branched —(C₁-C₃₀)alkylene-, and a biradical of formula (IV), (V), (VI), (VII), (VIII), (IX), (X), or (XI)

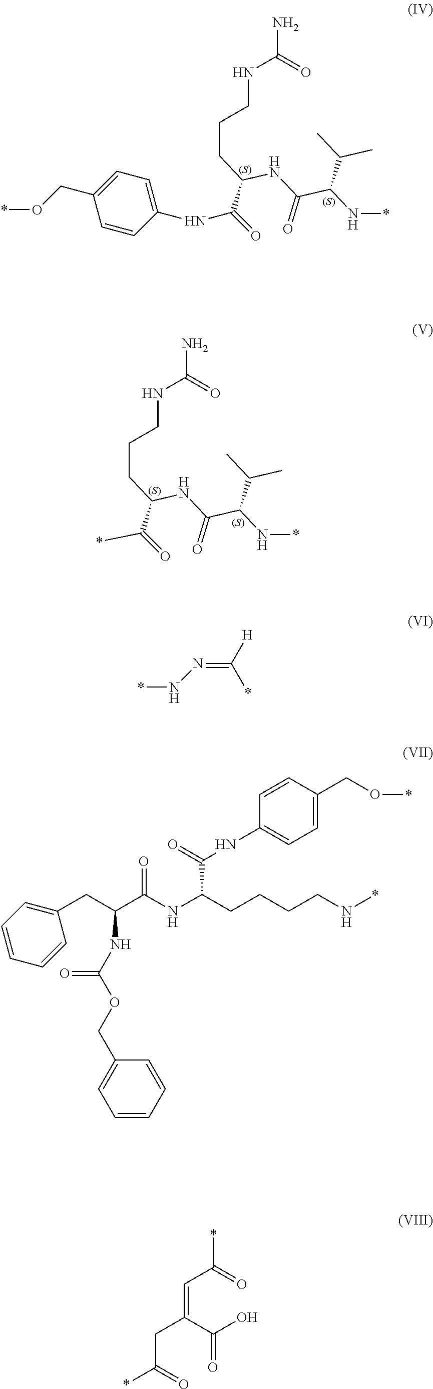

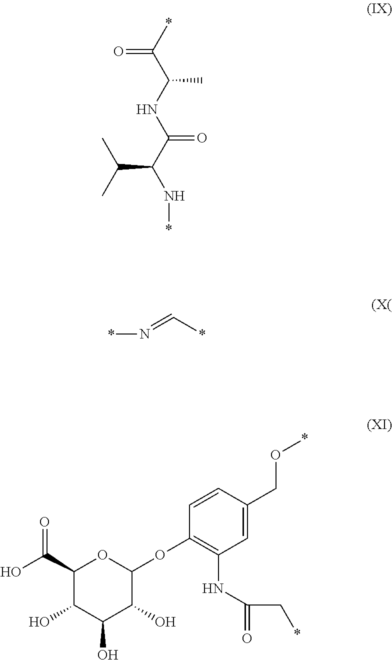

wherein the “*” denotes the attaching points;
wherein the —(C₁-C₃₀)alkylene biradical of Y is optionally substituted with one or more radical selected from the group consisting of —OH, —NRₐR_{b}, —SH₂, —NHNH₂, —COOR_{c}, —CF₃, —OCF₃, and halogen, wherein Rₐ, R_{b} and R_{c} are radicals independently selected from the group consisting of H, -phenyl, —(C₁-C₃₀)alkyl, —(C₂-C₃₀)alkenyl, —(C₁-C₃₀)alkylphenyl, and -phenyl(C₁-C₃₀)alkyl.

More preferably, R₁ is a biradical selected from the group consisting of —CH₂CH₂—S—S—CH₂CH₂—, —CH₂CH₂CH₂—S—S—CH₂CH₂CH₂—, —CH₂—, —CH₂CH₂—, —CH₂CH(CH₃) CH₂—, —CH₂CH(CH₂CH₃) CH₂—, —CH₂CH₂CH₂CH₂—, —CH₂COO—, —CH₂CH₂COO—, —CH₂CHCH₃COO—, —CH₂CH₂CH₃CH₂COO—, and a biradical selected from the group consisting of (IV), (V), (VI), (VII), (VIII), (IX), (X), (XI) and (XI) as defined herein.

Most preferably R₁ is ethylene.

Graft Copolymer of First Aspect—Lys/Orn Related Units with Integers “o” and “u”

In formula (I)—main repeating units defined by square brackets with the numerical values “o” and “u” may be seen as related to use of e.g. Lysine/Ornithine (Lys/Orn).

In an embodiment of the invention—the integer “o” is ≠0.

L may preferably be a single bond.

Orn may be suitable amino acid—i.e. it may be preferred that α is an integer of 2.

In several herein exemplified structures is used Lys—accordingly, it is preferred that xx is an integer of 3.

If the integer “o” is ≠0 and “u” is zero then it is understood that all Lys/Orn related repeating units are bound to branches with an amino acid structure of repeating units “p” and optionally also “q”.

Working examples herein describe structures where the majority of Lys/Orn related repeating units are bound to branches with an amino acid structure.

Accordingly, in a preferred embodiment the numerical value of integer “u” is less than 25% of the numerical value of integer “o”—i.e. if the numerical value of integer “o” is 100 then is the numerical value of integer “u” less than 25.

More preferably, the numerical value of integer “u” is less than 15% of the numerical value of integer “o”—even more preferably, the numerical value of integer “u” is less than 5% of the numerical value of integer “o”—and most preferably, the numerical value of integer “u” is less than 0.5% of the numerical value of integer “o”.

In several herein exemplified structures—the integer “o” is ≠0 and Glu/Asp related units with integers “r” and “v” not present—i.e. the integers “r” and “v” are zero.

Accordingly, in a preferred embodiment—the integer “o” is ≠0 and the integers “r” and “v” are 0.

Preferably, the numerical value of integer “o” is from 5 to 3000 (more preferably from 10 to 2500, even more preferably from 50 to 2000 and most preferably from 100 to 1500).

Preferably, the numerical value of integer “p” is from 3 to 1000 (more preferably from 5 to 750, even more preferably from 8 to 250 and most preferably from 10 to 200).

As illustrated in the “St-PLys-g-PHis-b-PGA” structure of FIG. 2—graft copolymer compound may also comprise an amino acid related block (b) structure—i.e. integer “q” is ≠0.

Accordingly, it may be preferred that the numerical value of integer “q” is from 3 to 1000 (more preferably from 5 to 750, even more preferably from 8 to 250 and most preferably from 10 to 200).

Preferably, each R₁₁ and R₁₂ is independently selected from H and a radical of formula (XII), (XIII), (XIV) and (XVI).

Preferably, R₁₁ is independently selected from H and a radical of formula (XII), (XIII), (XIV) and (XVI).

As discussed above and in working Examples herein, the novel graft copolymers discussed herein may have a molecular weight (MW) that is so high that a self-assembly process may not be required in order to make structures in the nanometer range (e.g. 4-300 nm in radius) that can be useful for delivery of e.g. active drug agents to target cells.

A high MW may be obtained by having a relatively high numerical value of integer “o” and integer “p”—accordingly in a preferred embodiment, the graft copolymer compound of the first aspect is a graft copolymer compound, wherein
the numerical value of integer “o” is from 25 to 5000 (more preferably from 100 to 2500) and the numerical value of integer “p” is from 2 to 2000 (more preferably from 10 to 500); and
the molecular weight (MW) is from 3 to 500000 kilodaltons (kDa)—more preferably from 10 to 100000 kilodaltons (kDa), even more preferably from 10 to 20000 kilodaltons (kDa), and most preferably from 20 to 10000 kilodaltons (kDa).

A preferred embodiment relates to a graft copolymer of the first aspect and herein relevant embodiments thereof, wherein the graft copolymer is a unimolecular structure with a radius from 4-300 nm.

Example 1 below describes examples of St-PLys-g-PGlu (see e.g. compounds 7A-7H) and provides data showing advantageous properties of these St-PLys-g-PGlu structures.

Accordingly, a preferred embodiment relates to such St-PLys-g-PGlu related compounds—i.e. wherein the integer “o” is ≠0, α is preferably an integer of 3 and R₁₁ is (XVI).

Example 1 below describes examples of graft copolymers designed to obtain unimolecular aggregates with hydrophobic pockets with the capability of encapsulating hydrophobic payloads-(see e.g. compounds 8-11).

Accordingly, a preferred embodiment relates to such “hydrophobic pockets” related compounds—i.e. wherein the integer “o” is ≠0, α is preferably an integer of 3 and R₁₁ is (XII) or (XIII).

To also have a block-P(hydrophilic aa) as in e.g. St-PLys-g-PBG-PGlu (compound 9) or St-PLys-g-PHis-PSar(compound 10)—it may be preferred that the integer “o” is #0, α is preferably an integer of 3, R₁₁ is (XII) or (XIII), integer “q” is ≠0, and R₁₂ is (XVI) or H.

Example 1 below describes examples of graft copolymers designed to obtain less compact structure such as e.g. St-(PLys-g-PGlu)-stat-PSar(compound 13).

Accordingly, a preferred embodiment relates to such “stat-PSar” related compounds—i.e. wherein the integer “o” is ≠0, α is preferably an integer of 3, R₁₁ is (XVI), integer “t” is ≠0 and R₁₅ is H.

Compound 14 of Example 1 is an example of conjugation of the hydrophobic drug Vitamin E (may herein be considered e.g. a cell-targeting agent).

Accordingly, a preferred embodiment relates to such “Vitamin E” related compounds—i.e. wherein “o” is ≠0 and R₁₁ is Vitamin E—preferably wherein α is preferably an integer of 3.

Compound 15 of Example 2 is an example of conjugation of a small molecule and compound 18 of Example 3 is an example of conjugation of therapeutic peptides.

Accordingly, a preferred embodiment relates to such conjugation related compounds—i.e. wherein the integer “o” is ≠0, a is preferably an integer of 3, R₁₁ and/or R₁₂ is (XIV), wherein Q of (XIV) is cell-targeting agent such as e.g. a small molecule (e.g. toll-like receptor agonist (TLR7/8) Imiquimod) or a therapeutic peptide (such as e.g. MHC-I and/or MHC-II).

Preferably, the graft copolymer of the first aspect is a compound comprising a compound selected from the group consisting of:
St-PLys-g-PGlu
St-PLys-g-PHis
St-PLys-g-PHis-b-PGlu
St-PLys-g-PHis-b-PSar
St-PLys-g-PBG
St-PLys-g-PBG-b-PGlu
St-PLys-g-PBG-b-PSar
St-PLys-g-PSar
St-PLys-g-PSar-g-VitE
St-(PLys-g-PGlu)-stat-PSar; and
St-PLys-b-PSar-g-PGA, and
wherein the term “St” is the star structure of formula (Ia) of the first aspect and/or embodiments thereof; and
wherein the term “PBG” is poly-Benzyl glutamate.

As understood by the skilled person in the present context—a compound comprising St-PLys-g-PHis will e.g. cover a compound comprising St-PLys-g-PHis-b-PGlu, since the latter also comprises St-PLys-g-PHis.

Similar—a compound comprising St-PLys-g-PHis will e.g. cover a compound comprising St-PLys-g-PHis and a conjugated agent of interest, since the latter also comprises St-PLys-g-PHis.

As discussed in e.g. working example below-PBG is poly-Benzyl glutamate.

Graft Copolymer of First Aspect—Glu/Asp Related Units with Integers “r” and “v”

In formula (I)—main repeating unit defined by square brackets with the numerical value “r” and “v” may be seen as related to use of e.g. Glutamic acid/Aspartic acid (Glu/Asp).

In an embodiment of the invention—the integer “r” is ≠0.

Asp may be suitable amino acid—i.e. it may be preferred that β is an integer of 0.

In several herein exemplified structures is used Glu—accordingly, it is preferred that β is an integer of 1.

If the integer “r” is ≠0 and “v” is zero then it is understood that all Glu/Asp related repeating units are bound to branches with an amino acid structure of repeating units “s”.

Working examples herein describe structures where a relevant amount of Glu/Asp related repeating units are bound to branches with an amino acid structure.

Accordingly, it may be relevant that the numerical value of integer “r” is at least 2% of the numerical value of integer “v”—i.e. if the numerical value of integer “v” is 100 then is the numerical value of integer “r” at least 2.

Alternatively—it may be relevant that the numerical value of integer “r” is at least 10% of the numerical value of integer “v”.

In several herein exemplified structures—the integer “r” is ≠0 and Lys/Orn related units with integers “o” and “u” not present—i.e. the integers “o” and “u” are zero.

Accordingly, in a preferred embodiment—the integer “r” is ≠0 and the integers “o” and “u” are 0.

Preferably, the numerical value of integer “r” is from 3 to 1000 (more preferably from 5 to 750, even more preferably from 7 to 500 and most preferably from 8 to 200).

Preferably, the numerical value of integer “s” is from 2 to 400 (more preferably from 3 to 350, even more preferably from 5 to 250 and most preferably from 10 to 200).

Preferably, R₁₃ is independently selected from H and a radical of formula (XII), (XIII), (XXI) and (XXVIII).

It may be preferred that each R₁₄ is independently selected from H, —OH.

As discussed above and in working Examples herein, the novel graft copolymers discussed herein may have a molecular weight (MW) that is so high that a self-assembly process may not be required in order to make structures in the nanometer range (e.g. 4-300 nm in radius) that can be useful for delivery of e.g. active drug agents to target cells.

A high MW may be obtained by having a relatively high numerical value of integer “r” and integer “s”—accordingly in a preferred embodiment, the graft copolymer compound of the first aspect is a graft copolymer compound, wherein
the numerical value of integer “r” is from 3 to 1000 (more preferably from 5 to 750) and the numerical value of integer “s” is from 2 to 400 (more preferably from 3 to 350); and
the molecular weight (MW) is from 3 to 500000 kilodaltons (kDa)—more preferably from 10 to 100000 kilodaltons (kDa), even more preferably from 10 to 20000 kilodaltons (kDa), and most preferably from 20 to 10000 kilodaltons (kDa).

A preferred embodiment relates to a graft copolymer of the first aspect and herein relevant embodiments thereof, wherein the graft copolymer is a unimolecular structure with a radius from 4-300 nm.

Example 7 below describes examples of graft copolymers with St-PGlu-g-PSar (compounds 19) or St-PGlu-g-PLysTG structures (compounds 20).

Accordingly, a preferred embodiment relates to such “compounds—i.e. wherein the integer “r” is ≠0, β is preferably an integer of 1, and R₁₃ is H or (XXI).

Example 8 below describes examples of graft copolymers with St-PGA-g-PSar-g-PHis (alternatively termed St-PGlu-g-PSar-g-PHis) related structures (compounds 21)

Accordingly, a preferred embodiment relates to such “compounds—i.e. wherein the integer “r” is ≠0, β is preferably an integer of 1, and R₁₃ is H and/or (XII).

Example 10 below describes examples of conjugation of hydrophobic drugs to St-PGlu-g-Psar (compounds 22).

Accordingly, a preferred embodiment relates to such “compounds—i.e. wherein the integer “r” is ≠0, “v” is ≠0, β is preferably an integer of 1, R₁₃ is H, R₁₄ is a cell-targeting agent (such as e.g. a hydrophobic drug such as e.g. glycine-vitamin E).

Preferably, the graft copolymer of the first aspect is a compound comprising a compound selected from the group consisting of:
St-PGlu-g-PSar
St-PGlu-g-PHis
St-PGlu-g-PLys
St-PGlu-g-PLysTG; and
St-PGlu-g-PSar-g-PHis; and
wherein the term “St” is the star structure of formula (Ia) of the first aspect and/or embodiments thereof; and
wherein PLysTG is poly-lysine-thioglycerated.

As understood by the skilled person in the present context—a compound comprising St-PGlu-g-PSar will e.g. cover a compound comprising St-PGlu-g-PSar-g-PHis, since the latter also comprises St-PGlu-g-PSar.

Similar—a compound comprising St-PGlu-g-PSar will e.g. cover a compound comprising St-PGlu-g-PSar and a conjugated agent of interest, since the latter also comprises St-PGlu-g-PSar.

As discussed in e.g. working example below-PLysTG is poly-lysine-thioglycerated.

Graft Copolymer of First Aspect—Ala/Sar Related Units with Integer “t”

In formula (I)—main repeating unit defined by square brackets with the numerical value “t” may be seen as related to use of e.g. Alanine/Sarcosine/Glycine (Ala/Sar/Gly).

Several working examples herein describe structures where the integer “t” is zero-accordingly, it may be preferred that the integer “t” is 0.

It may be preferred that the numerical value of integer “t” is from 1 to 5000 (more preferably from 3 to 4000.

### Graft Copolymer of First Aspect—Other Matter

It may be preferred that:
each R₃ is independently selected from —H;
each R₄ is independently selected from —H;
each R₅ is independently selected from —H;
each R₆ is independently selected from —CH₃,
each R₇ is independently selected from —H;
each R₈ is independently selected from —CH₃;
each R₉ is independently selected from —H;
each R₁₀ is independently selected from CH₃;
each R₁₅ is independently selected from H;

It may be preferred that each X₁ is independently selected from H, —C(O)H, —S(O)H, —NHCH(CH₃)₂, —(C₁-C₄)alkylNH₂, —(C₁-C₄)alkylNHCH₃, —(C₁-C₄)alkylN(CH₃)₂, —O—(C₁-C₄)alkyl-NH₂, —O—(C₁-C₄)alkyl-NHCH₃, and —O—(C₁-C₄)alkyl-N(CH₃)₂;

It may be preferred that each X₂ is independently selected from H, —C(O)H, —S(O)H, —NHCH(CH₃)₂, —(C₁-C₄)alkylNH₂, —(C₁-C₄)alkylNHCH₃, —(C₁-C₄)alkylN(CH₃)₂, —O—(C₁-C₄)alkyl-NH₂, —O—(C₁-C₄)alkyl-NHCH₃, and —O—(C₁-C₄)alkyl-N(CH₃)₂.

It may be preferred that each X₃ is independently selected from H, —C(O)H, —S(O)H, —NHCH(CH₃)₂, —(C₁-C₄)alkylNH₂, —(C₁-C₄)alkylNHCH₃, —(C₁-C₄)alkylN(CH₃)₂, —O—(C₁-C₄)alkyl-NH₂, —O—(C₁-C₄)alkyl-NHCH₃, and —O—(C₁-C₄)alkyl-N(CH₃)₂.

### A Polymer Complex of Second Aspect

As discussed above, a second aspect of the invention relates to a polymer complex comprising a graft copolymer of the first aspect and/or embodiment thereof, and at least a pharmaceutical, veterinary or cosmetical active agent.

Preferably, the at least one pharmaceutical, veterinary or cosmetical active agent is selected from the group consisting of low molecular weight drugs, peptides, proteins, antibodies, nucleic acids, aptamers, and combinations thereof.

The nucleic acid may e.g. be selected from the group consisting of plasmid DNA, CIDNA, siRNA, mRNA, microRNA, donorDNA, sgDNA, crDNA, shRNA, antisense nucleic acid, a decoy nucleic acid, an aptamer, and a ribozyme.

As known in the art—an active agent of interest may be covalently linked to a polypeptidic backbone—e.g. through an amino acid side residue via amide, ester, anhydride bonding or through a linker that includes one or more functional groups, including without limitation, alkynes, azides, reactive disulfides, maleimides, hydrazide, hydrazones, Schiff bases, acetal, aldehydes, carbamates, and reactive esters. In an alternative embodiment the covalent link is a bioresponsive one.

As known in the art—an active agent of interest may be linked to a polypeptidic backbone through electrostatic interaction. Thus, anionic compounds having more negative charges than positive charges may form a polymer complex with a graft copolymer of the first aspect when mixed in aqueous medium, through electrostatic interaction. Examples of anionic compounds include proteins, polysaccharides, lipids and nucleic acids.

As known in the art—an active agent of interest may be linked to a polypeptidic backbone through e.g. hydrophobic and/or hydrophilic related interactions.

For instance, Example 1 below describes examples of graft copolymers designed to obtain unimolecular aggregates with hydrophobic pockets with the capability of encapsulating hydrophobic payloads (e.g. active agents).

Accordingly, based on common general knowledge and the technical disclosure herein—it is routine work for the skilled person to make a polymer complex of the second aspect and/or embodiment thereof of the present invention.

It may be preferred that a polymer complex of the second aspect is a polymer complex, wherein:
—the active agent is covalently linked to a polypeptidic backbone of the polymer complex;
—the active agent is linked to a polypeptidic backbone of the polymer complex through electrostatic interaction; or
the active agent is linked to a polypeptidic backbone of the polymer complex through hydrophobic and/or hydrophilic related interactions.

### A Pharmaceutical, Veterinary or Cosmetic Composition of Third Aspect

As discussed above, a third aspect of the invention relates to a pharmaceutical, veterinary or cosmetic composition comprising at least one polymer complex of the second aspect and/or embodiment thereof, together with one or more appropriate excipients or carriers.

Based on common general knowledge and the technical disclosure herein—it is routine work for the skilled person to make pharmaceutical, veterinary or cosmetic composition of the third aspect and/or embodiment thereof of the present invention.

As known in the art-pharmaceutical, veterinary or cosmetic composition also comprises acceptable excipients—i.e. pharmaceutical, veterinary or cosmetic acceptable excipients.

### Medical Use—Fourth and Fifth Aspect

As discussed above, a fourth aspect of the invention relates to a polymer complex of the second aspect and/or embodiment thereof, or the pharmaceutical composition according of the third aspect and/or embodiment thereof, for use as a medicament.

As discussed above, a fifth aspect of the invention relates to a polymer complex of the second aspect and/or embodiment thereof, or the pharmaceutical composition according of the third aspect and/or embodiment thereof, for use (i) as transfection reagent for transfecting at least one active agent into a cell; (ii) for use in the in vivo or ex vivo production of biologics encoding a recombinant protein, a peptide or an antibody, or in the production of recombinant virus; (iii) for use as a therapeutic or prophylactic vaccine against viral infections or as a therapeutic vaccine against cancers; or (v) for use in genome engineering, for cell reprogramming, for differentiating cells or for gene-editing.

Based on common general knowledge and the technical disclosure herein—it is routine work for the skilled person to identify a suitable medical use a polymer complex and/or a pharmaceutical composition described herein.

For instance, many of the medical uses described in the art for related polymer compounds (see e.g. above discussed EP3331937B1) may also be relevant for the graft copolymer compounds of the present invention.

Delivering a Pharmaceutical Active Agent into a Target Cell—Sixth Aspect

As discussed above, a sixth aspect of the invention relates to a method for delivering a pharmaceutical active agent (e.g. a nucleic acid) into a target cell, which comprises administering a composition (e.g. solution) that contains the polymer complex of the second aspect and/or embodiment thereof to an animal, including human, so that the complex is getting into physical contact with the target cell and thereby delivers the pharmaceutical active agent into the cell.

Based on common general knowledge and the technical disclosure herein—it is routine work for the skilled person to identify a suitable method to deliver an active agent of interest to a target cell of interest.

As understood by the skilled person—the term “the complex is getting into physical contact with the target cell” may e.g. relate to that the complex is introduced into the target cell.

Alternatively, the complex may e.g. be bound to the surface of the target cell and the active agent is delivered into the cell due to it e.g. crosses the surface cell membrane and thereby gets into the cell.

Working examples herein describe that graft copolymer compounds of the present invention may get into physical contact with a target cell of interest and thereby delivers an active agent of interest into the cell.

Further, related polymer compounds (see e.g. above discussed EP3331937B1) are also described as being useful for delivering an active agent into a target cell of interest.

### Use of Graft Copolymer of Invention to Make a Different Compound—Seventh Aspect

As discussed above, a seventh aspect of the invention relates to a process for preparing a compound which is structurally different from a graft copolymer starting compound of the first aspect and/or embodiment thereof, comprising following steps:
(i): using a compound of the first aspect and/or embodiment thereof as a starting compound;
(ii) making structural changes to the compound of step (i) to obtain a compound, which is structurally different from the starting compound of (i).

Preferably, the obtained structurally different compound of step (ii) is a compound that has similar functions/capabilities as the starting compound of step (i)—i.e. it is preferred that the obtained structurally different compound of step (ii) is a compound which is capable of forming a polymer complex comprising the obtained structurally different compound of step (ii), and at least a pharmaceutical, veterinary or cosmetical active agent.

Preferably, the obtained structurally different compound of step (ii) is a compound, which is capable of forming a polymer complex comprising the obtained structurally different compound of step (ii), and at least one pharmaceutical, veterinary or cosmetical active agent, and wherein the active agent is an active agent that the starting compound of step (i) is also capable of forming a polymer complex with.

The obtained structurally different compound of step (ii) may e.g. be a compound outside the scope of protection of a graft copolymer compound of the first aspect and/or embodiment thereof.

Alternatively, the obtained structurally different compound of step (ii) it a compound within the scope of protection of a graft copolymer compound of the first aspect and/or embodiment thereof.

Based on common general knowledge and the technical disclosure herein—it is routine work for the skilled person to use a graft copolymer compound as described herein as a starting compound to make a structurally different compound.

As understood by the skilled person—the process of the seventh aspect is based in the technical disclosure herein—since it uses a graft copolymer compound of the first aspect (i.e. of the present invention) to make the structurally different compound.

### Process for the Synthesis of Compound of the First Aspect—Separate Aspect

A separate aspect of the invention relates to a process for the synthesis of a graft copolymer compound of the first aspect and/or embodiment thereof, the process generally comprising polymerizing N-carboxy anhydrides (NCA) of protected or non-protected amino acids known per se with a 3-arm star initiator (St); followed by a deprotection reaction, and then a second, and optionally additional, polymerization reactions.

In accordance with this aspect, the 3-arm star initiator (St), N1,N3,N5-tris(2-aminoethyl)benzene-1,3,5-tricarboxamide (1) (i.e. St-initiator (1)), may be generally obtained by reacting 1,3,5-benzenetricarbonyl trichloride with N,N′-Diisopropylethylenediamine (DIEA) to obtain the intermediate 1,3,5-tri-tert-butyl ((benzenetricarbonyltris-(azanediyl))tris(ethane-2,1-diyl))tricarbamate (2); intermediate (2) is then reacted with TFA to obtain 1,3,5 (benzenetricarbonyltris(azanediyl))-triethanamonium TFA salt (3) (i.e. star polymer TFA protected intermediate (3)); TFA groups are then removed by a cleavage reaction with basic deprotection (e.g. by stirring in an appropriate ion-exchange resin) to obtain St-initiator (1).

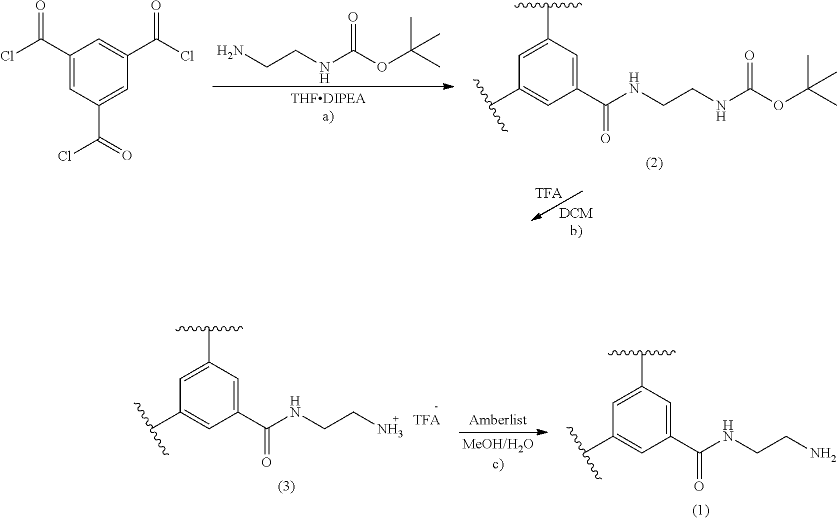 [SMILES: O=C(Cl)c1cc(C(=O)Cl)cc(C(=O)Cl)c1 ; CC(C)(C)OC(=O)NCCN ; *c1cc(*)cc(C(=O)NCCNC(=O)OC(C)(C)C)c1 ; *c1cc(*)cc(C(=O)NCCN)c1 ; *c1cc(*)cc(C(=O)NCCN)c1]

In accordance with this aspect, related to a process for the synthesis of the compound of formula (I) of the first aspect and/or embodiment thereof, the process generally comprising:
i) polymerizing N-carboxy anhydrides (NCA) of protected (e.g. tetrafluoroborate or trifluoroacetate ammonium salt form) or non-protected amino acids known per se with a 3-arm star initiator, St-initiator (1) or the St-protected initiator intermediate (3), in a sequential or statistical manner to obtain a block co-polymer or alternatively a random co-polymer;
ii) optionally, reacting the amine group at the N-terminal position with an amine reactive group to introduce an end-capping group;
iii) optionally, orthogonally removing amino acid side chain protecting groups;
iv) optionally, reacting the amine group at side chain terminal position with an amine reactive group to introduce architectural extension, conjugation, labelling or shielding;
v) purifying the product obtained in step i), ii), iii), or iv) optionally by fractionation, precipitation, ultrafiltration, dialysis, size-exclusion chromatography, affinity chromatography or tangential flow filtration.
Step i) above may include: a) ring opening polymerization of non-protected amino acid N-carboxyanydride (NCA) monomer by reacting the tetrafluoroborate or trifluoroacetate ammonium salt form of St-protected initiator intermediate (3) with the selected non-protected NCA-amino acid monomer; or alternatively ring opening polymerization of protected amino acid N-carboxy anhydrides (NCA) monomer (e.g. tetrafluoroborate or trifluoroacetate ammonium salt form) by reacting the St-initiator (1) with the selected protected NCA-amino acid monomer; wherein the ratio monomer/initiator allows the control of the degree of polymerization (DP). The polymerization with different amino acid NCA monomers may be performed in a sequential manner, i.e. wherein block co-polypeptides are prepared following the polymerization reaction a) in a sequential manner, allowing the first NCA monomer to be consumed and the resulting product may be purified or not before adding the next NCA-monomer to build the following polypeptidic block; or alternatively in a statistical manner wherein random copolypeptides are prepared following the polymerization reaction a) in a statistical manner, mixing all the NCA-monomers before starting the polymerization.
Step ii) above corresponds to the end-capping, wherein the amine group at the N-terminal position is reacted with an amine reactive group to introduce an end-capping functional group.
Step iii) above corresponds to the deprotection, wherein amino acid side chains are removed orthogonally depending on the protecting group.
Step iv) corresponds to the conjugation, reacting the amine group at side chain terminal position of a shielding polymer, an active small molecule, a targeting agent or an imaging agent with an amine reactive group.

### EXAMPLES

### Analytical Methods

NMR spectroscopy: NMR spectra were recorded at 27° C. (300 K) on a 300 Ultrashield™ from Bruker (Billerica MA, USA). Data were processed with the software Topspin (Bruker GmbH, Karlsruhe, Germany). Samples were prepared at a concentration of 20-10 mg/mL approx. in the required solvent.

Size Exclusion Chromatography (SEC): For SEC measurements in dimethylformamide (DMF) containing 0.1% (w/w) of lithium bromide as an additive, a GPC max (Malvern Instruments) autosampler was used with a flow rate of 0.7 mL/min at 60 TSKgel Alpha-4000 column from Tosoh 0 with Bioscience. Viscotek TDA-302 was used as an integrated detection system. The system was calibrated with polymethyl methacrylate (PMMA) (Mw=65 kDa; PDI=1.05) from PSS. For this Mw determination an integrated triple detection system was used [Refractive Index, Light Scattering (two angles: 7 and 90°) and Ultraviolet-Visible detector].

For SEC measurements in aqueous containing 0.1M of sodium nitrate and 0.005% of sodium azide as an additive, a GPC max (Malvern Instruments) pump and autosampler was used with a flow rate of 0.7 mL/min at 25° C. with TSKGel PWXL G5000 column from Tosoh Bioscience. Viscotek TDA-305 was used as an integrated detection system. The system was calibrated with polyethylene oxide (PEO) (Mw=24 kDa; PDI=1.01) from Malvern Panalytical. For this Mw determination an integrated triple detection system was used [Refractive Index, Light Scattering (two angles: 7 and 90°) and Ultraviolet-Visible detector].

Size Distribution. Dynamic Light Scattering (DLS) measurements were performed using a Malvern Zetasizer NanoZS instrument, equipped with a 532 nm laser at a fixed scattering angle of 173°. Polymer solutions were prepared under different conditions (MilliQ or PBS at different concentrations and temperatures), solutions were sonicated for 10 min and allowed to age for the required time, filtered through a 1.20 μm cellulose membrane filter and measured. Size distribution was measured (diameter, nm) for each polymer per triplicate with n>3 measurements, automatic optimization of beam focusing and attenuation was applied for each sample.

### Example 1. Preparation of Compounds of Formula (I)—Lys Bound to Branches with Different Amino Acid Structures

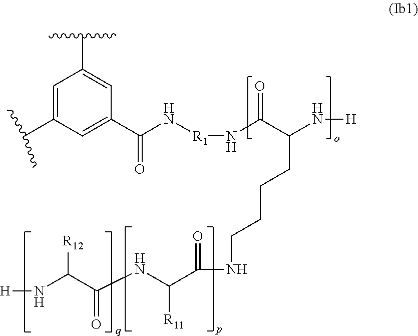

In relation to formula (Ia) of the first aspect herein-formula (Ib1) above is an example, wherein the integer “o” is ≠0 and α is an integer of 3—i.e. relating to use of Lys to bind branches with different amino acid structures.

In general terms, to synthesize compounds of formula (Ib1) according to the present disclosure, first, the 3-arm star initiator was obtained within 2-3 steps. Such initiator was used to polymerize N-Trifluoroacetyl-L-lysine N-carboxyanhydride or Lysine (TFA) NCA, to yield the star polymer TFA protected (St-PLys(TFA)). The TFA groups were removed by a cleavage reaction with basic deprotection to yield the corresponding Star-PLys with free NH₂ active sites to initiate the next polymerization steps.

Scheme 1 shows a particular example of polymerization, deprotection steps and a grafting from approach from a Poly-L-Lysine main chain.

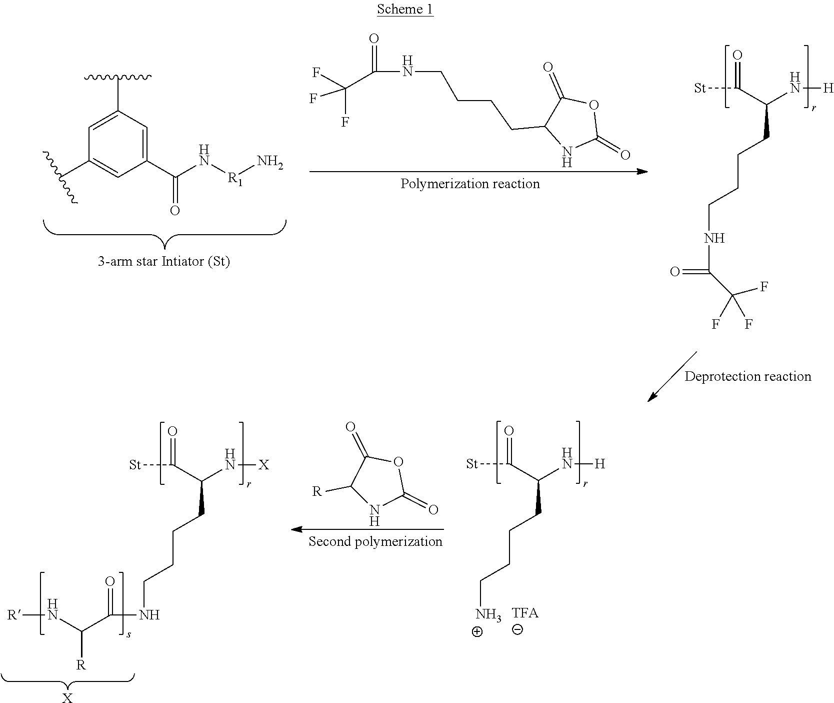

### Example 1A: Synthesis of 3-Arm Star Initiator

The synthetic route towards the 3-arm star initiator, N1,N3,N5-tris(2-aminoethyl)benzene-1,3,5-tricarboxamide (1) is disclosed in Scheme 2.

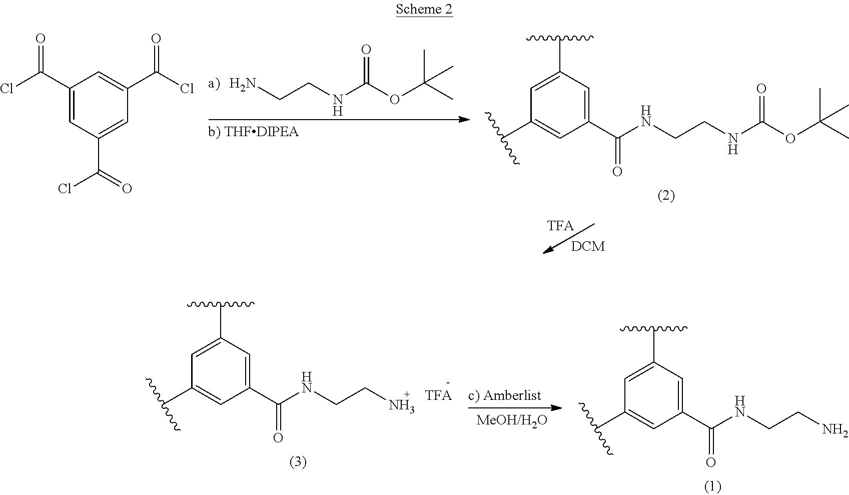 [SMILES: O=C(Cl)c1cc(C(=O)Cl)cc(C(=O)Cl)c1 ; CC(C)(C)OC(=O)NCCN ; *c1cc(*)cc(C(=O)NCCNC(=O)OC(C)(C)C)c1 ; *c1cc(*)cc(C(=O)NCCN)c1 ; *c1cc(*)cc(C(=O)NCCN)c1]

### Step (a): Synthesis of 1,3,5-tri-tert-butyl ((benzenetricarbonyltris-(azanediyl))tris(ethane-2,1-diyl))tricarbamate (2)

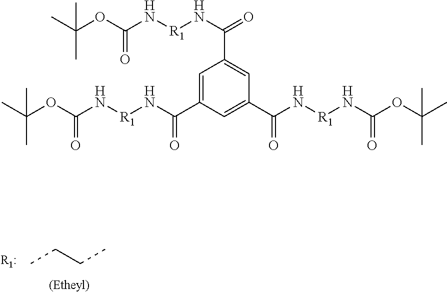

In a two-neck round bottom flask fitted with a stirrer bar, and a N2 inlet and outlet, 500 mg of 1,3,5-benzenetricarbonyl trichloride (1.88 mmol, 1 equivalent) was dissolved in 12 mL of anhydrous THF. N,N′-Diisopropylethylenediamine (DIEA) (803.31 mg, 6.22 mmol, 3.3 eq.) was added to the reaction mixture followed by drop-wise addition of N-Boc-ethylenediamine (1.34 g, 6.22 mmol, 3.3 eq.) over a period of 10 min. The reaction was then left to proceed for 2 hours. After that time, the solvent was completely removed under vacuum. The product was re-dissolved in chloroform and washed 3 times with deionized water (ddH₂O), and 3 times with acidic water (PH˜3).

Finally, the organic phase was isolated under vacuum and the product was recrystallized 3 times from THF/Methanol/Hexane yielding a white crystalline solid. The product was then dried under high vacuum and stored at −20° C.

Yield: 82%. ¹H NMR (300 MHZ, DMSO) δ 8.68-8.65 (m, 3H), 8.41 (s, 3H), 6.92-6.88 (m, 3H), 3.34-3.31 (m, 6H), 3.16-3.13 (m, 6H), 1.37 (s, 27H). ¹³C NMR (75 MHz, CDCl₃) δ 166.80 (C═O), 156.84 (C═O), 134.58 (CAr quaternary), 128.47 (CH_{Ar}), 79.57 (C quaternary), 40.93 (CH₂), 40.43 (CH₂), 28.45 (CH₃).

### Step (b): Synthesis of 1,3,5-(benzenetricarbonyltris(azanediyl))-triethanamonium TFA Salt (3)

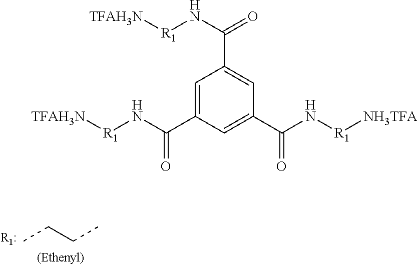

In a round bottom flask fitted with a stirrer bar and a stopper, 200 mg of 1,3,5-tri-tert-butyl ((benzenetricarbonyltris(azanediyl)) tris(ethane-2,1-diyl)) tricarbamate (2) (0.33 mmol, 1 eq.) was dissolved in 5 mL of anhydrous dichlorometane and 2.5 mL of TFA was added. The reaction was stirred under nitrogen atmosphere for 2 hours and the completion of the reaction was monitored by TLC. The solvents were evaporated in vacuo. The TFA salt of the initiator (220 mg) was obtained in quantitative yield and dried under vacuum.

Yield: 98%. ¹H NMR (300 MHZ, D₂O) δ 8.36 (s, 3H), 3.75 (t, J=5.9 Hz, 6H) 3.29 (t, J=6.0 Hz, 6H). ¹⁹F NMR (300 MHz, D₂O) δ−75.84.

### Step (c): Synthesis of N¹,N³,N⁵-tris(2-aminoethyl)benzene-1,3,5-tricarboxamide (1)

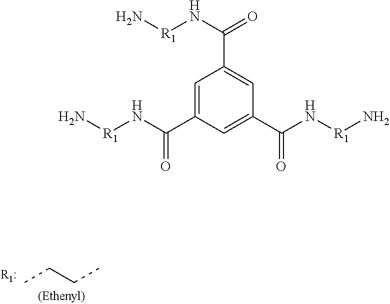

220 mg of 1,3,5-(benzenetricarbonyltris(azanediyl)) triethanamonium TFA salt (3) was dissolved in 22 mL of the mixture H₂O: MeOH (7:3), and stirred with an excess of the weakly basic Amberlyst ion-exchange resin (1000 mol %) for 24 hours. Then, the mixture was filtered and the filtrate was concentrated by removing the methanol. The aqueous solution was lyophilized obtaining the freebase amine in high purity. Yield: 98%. ¹H NMR (300 MHZ, D₂O) δ 8.13 (s, 3H), 3.45 (t, J=6.3 Hz, 6H) 2.86 (t, J=6.3 Hz, 6H).

### Example 1B: Polymerization of Main Chain

The synthesis of compound (5) is performed following the synthetic route of Scheme 3.

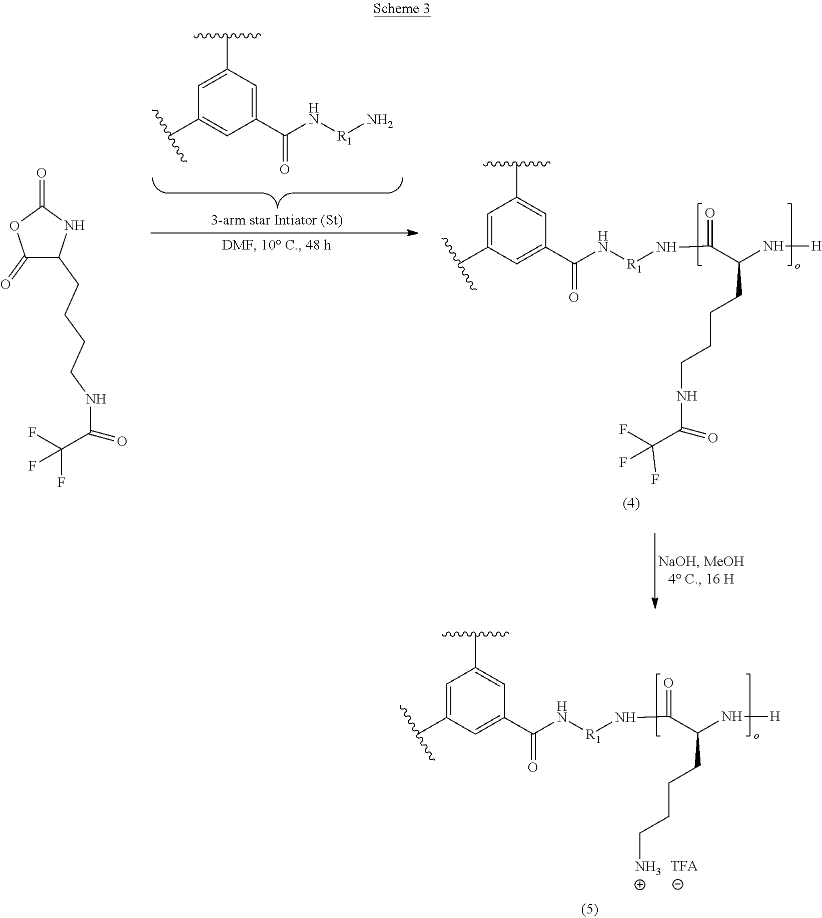

Briefly, L-Lysine (TFA)-NCA (2.0 g, 7.46 mmol) was added to a Schlenk tube fitted with a stirrer bar, a stopper and purged with 3 cycles of vacuum/N2, and dissolved in an anhydrous DMF (4 mL). Then, the star initiator (St) (16.72 mg, 0.05 mmol) was dissolved in DMF (1 mL) and was added to the reaction mixture. The mixture was stirred at 10° C. for 48 hours. Upon completion full conversion of the monomer could be detected by IR. The reaction mixture was poured into diethyl ether (1:10 ratio) to precipitate the product. The precipitate was isolated by centrifugation (3750 rpm, 4 min) and dried under vacuum. Star-Poly(L-Lys(TFA)) (St-PLys(TFA)) (4) was isolated as a white solid. In a second step, the TFA protecting group from St-PLys(TFA) (4) is deprotected under basic conditions. Briefly, the polymer (4) (0.45 g, 2.00 mmol) is dissolved in MeOH (1.5 mL) and NaOH 4.5 M (aq) (0.67 mL) were added to the reaction mixture. The mixture was stirred at 4° C. for 16 hours. After that time, TFA 5 M (aq) (0.55 mL) the solution was poured into acetonitrile (1:10 ratio) to precipitate the product. The precipitate was isolated by centrifugation (3750 rpm, 4 min) and dried under vacuum. Star-Poly(L-Lys) (St-PLys) (5) was isolated as a white solid.

Yield: 70-90% ¹H NMR (D₂O): δ=1.46 (m, 2H, CH₂), 1.64-1.90 (broad m, 4H, CH₂, CH₂), 3.03 (t, 2H, CH₂—NH₂), 3.34-3.66 (broad m, 12H, CH₂—CH₂), 4.33 (t, 1H, quiral CH), 8.31 (s, 3H, aryl CH).

Table 1 Shows different DPs (degree of polymerization) obtained for different St-PLys of formula (5), demonstrating the versatility and accuracy of the experimental procedure.

TABLE 1

| Com- | | | Mn^{a} | Mn^{b} | | | |
|---|---|---|---|---|---|---|---|
| pound | R1 | DPLys^{theo} | (kDa) | (kDa) | DP^{a} | DP^{b} | Ð |
| 5A | —CH₂CH₂— | 75 | 9.2 | 9.5 | 69 | 71 | 1.02 |
| 5B | —CH₂CH₂— | 150 | 18.7 | 15.4 | 142 | 136 | 1.08 |
| 5C | —CH₂CH₂— | 300 | 41.0 | 37.1 | 315 | 330 | 1.05 |
| 5D | —CH₂CH₂— | 600 | 82.6 | 66.3 | 638 | 620 | 1.03 |
| 5E | —CH₂CH₂— | 1000 | 144.3 | 168.2 | 1116 | 1301 | 1.73 |

^{a}Determined by NMR.
^{b}Determined by SEC.
Mn and DP refer to number average molar mass and degree of polymerization respectively.
Ð represent polydispersity as determined by SEC-MALS software analysis.

### Example 1C: Grafting from

Synthesis of Star-Poly(L-Lys) graft-Poly(L-PGlu), (St-PLys-g-PGlu) (7). Scheme 4 shows the synthetic route to obtain compound 7.

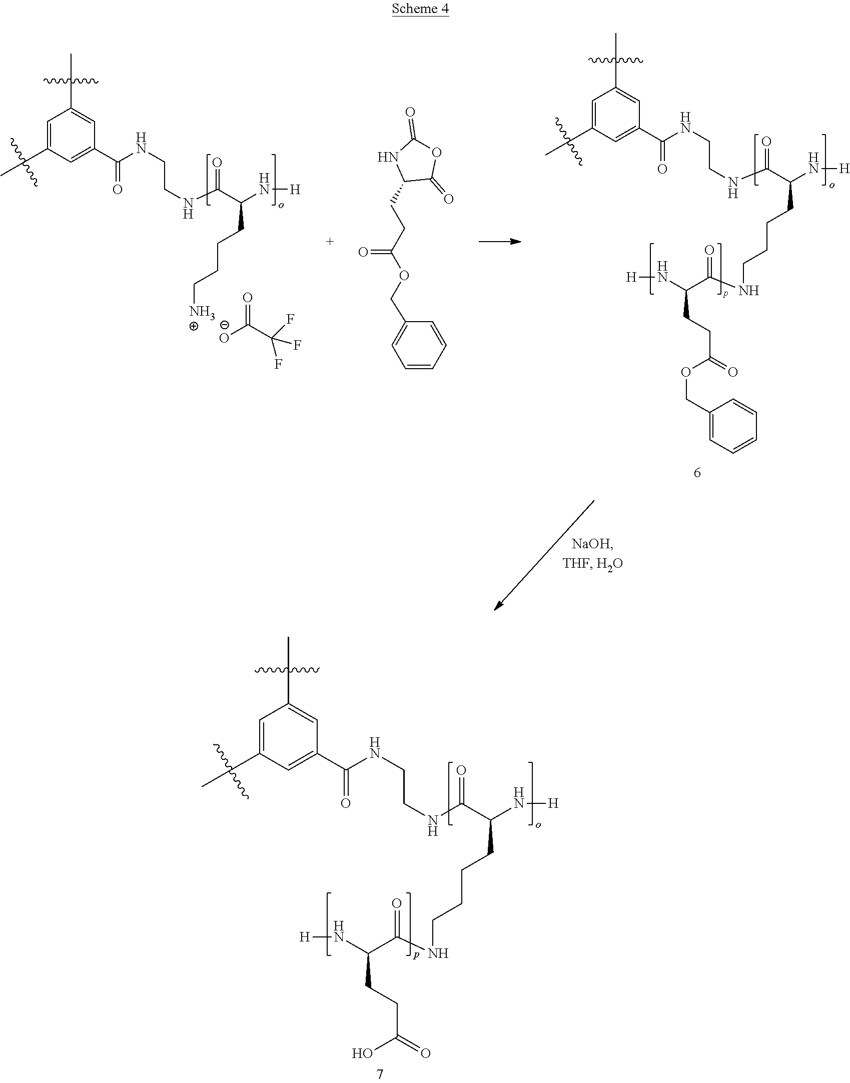

γ-Benzyl L-glutamate N′carboxyanhydride (99.1 g, 0.38 mol) was added to a Schlenk tube fitted with a stirrer bar, a stopper and purged with 3 cycles of vacuum/Ar, and dissolved in 490 mL of anhydrous DMF. The required equivalents of compound (5) (837 mg, 3.45 mmol) as macroinitiator to graft from were dissolved in DMF (10 mL), added and the mixture was left stirring at r.t for 2 days under inert atmosphere. Upon completion full conversion of the monomer could be detected by IR. Finally, the reaction mixture was poured into a large excess of cold diethyl ether (1:10 ratio) leading to the white polymer Star-Poly(L-Lys)-graft-Poly(L-PBG), (St-PLys-g-PBG) (6) after isolation by filtration or centrifugation (3750 rpm, 4 min) and dried under vacuum. Following this strategy, the degree of polymerization was controlled by the feeding ratio [monomer]/[macroinitiator] to yield graft copolymers. In a second step, the benzyl protecting group from St-PLys-g-PBG (6) is deprotected under basic conditions. Briefly, the polymer (6) (69.1 g, 0.32 mol) is dissolved in THF (2.8 L) and NaOH 0.7 M (aq) (0.7 L) were added to the reaction mixture. The mixture was stirred at 0° C. for 16 hours. After that time, the solution was poured into a mixture of diethyl ether/acetonitrile (3/1) to precipitate the product. The precipitate was isolated by centrifugation (3750 rpm, 4 min) and dried under vacuum. Star-Poly(L-Lys)-graft-Poly(L-PGlu), (St-PLys-g-PGlu) (7) was isolated as a white solid.

Yield: 70-90% ¹H NMR (TFA): δ=2.92 (m, 2H, CH₂), 4.85 (s, 1H, CH), 5.05 (m, 2H, benzyl CH₂), 7.13 (s, 5H, aryl CH), 8.38 (s, aryl CH).

Table 2 Shows different DPs (degree of polymerization) obtained for different St-PLys-g-PGlu of formula (7), demonstrating the versatility and accuracy of the experimental procedure.

TABLE 2

| | DP | Graft | Graft | Graft | MW | | GE* |
|---|---|---|---|---|---|---|---|
| Compound | PLys | polymer | DP^{theo} | DP^{a} | (MDa)^{a} | Ð^{a} | % |
| 7A | 75 | PGlu | 110 | 51 | N/A | N/A | 46 |
| 7B | 136 | PGlu | 110 | 60 | 1.18 | 1.05 | 55 |
| 7C | 300 | PGlu | 30 | 25 | 1.40 | 1.06 | 84 |
| 7D | 600 | PGlu | 15 | 13 | 1.41 | 1.07 | 90 |
| 7E | 1000 | PGlu | 9 | 8 | 1.37 | 1.13 | 87 |
| 7F | 300 | PGlu | 110 | 44 | 1.79 | 1.01 | 41 |
| 7G | 600 | PGlu | 110 | 36 | 2.70 | 1.01 | 34 |
| 7H | 1000 | PGlu | 110 | 31 | 3.20 | 1.02 | 29 |

^{a}Determined by SEC.
Mn and DP refer to number average molar mass and degree of polymerization respectively.
Ð represent polydispersity as determined by SEC-MALS software analysis.
*GE: grafting efficiency.

### Example 1D: Star-Poly(L-Lys)-graft-Poly(L-hydrophobic aa)-block-P(hydrophillic aa), Compounds 8-11

This graft copolymers were designed in order to obtain unimolecular aggregates with hydrophobic pockets with the capability of encapsulating hydrophobic payloads. Scheme 5 shows a general synthetic scheme to obtain compounds from 8-11.

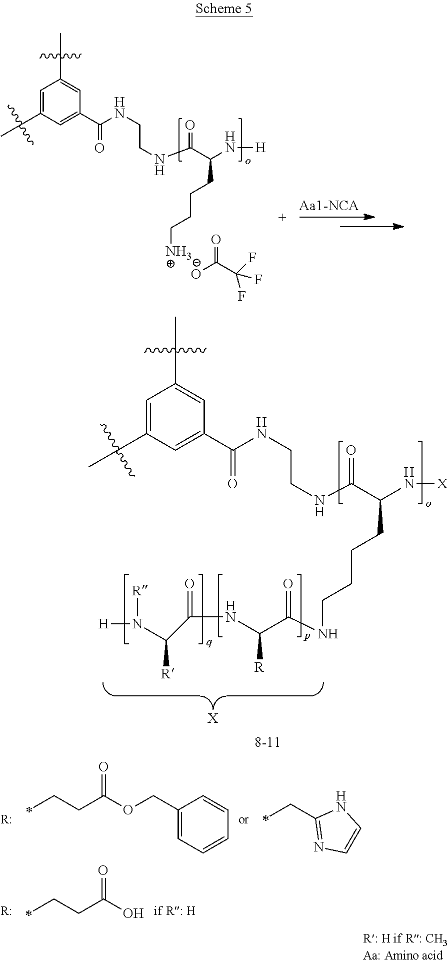

Similar to the synthesis of compound 6, the macroinitiator St-PLys(5) was used to polymerize a first and then a second amino acid NCA. In the particular cases of this example, the amino acid NCA-1 was either y-benzyl-L-glutamate NCA or N(Im)-(2,4-Dinitrophenyl)-L-histidine NCA (His(DNP)NCA). In brief, the particular NCA-1 was added to a Schlenk tube fitted with a stirrer bar, a stopper and purged with 3 cycles of vacuum/Ar, and dissolved in anhydrous DMF. The required equivalents of compound (5) as macroinitiator to graft from were added and the mixture was left stirring at 10° C. for 3-4 hours under inert atmosphere. Upon completion full conversion of the monomer could be detected by IR. Next, the second monomer was added to polymerize a second block in one-pot. In the particular cases of this example, the amino acid NCA-2 was either sarcosine NCA added in anhydrous DMF or Glutamic acid 5-tert-butyl ester NCA (Glu(OtBu)NCA) added in anhydrous THF. Upon completion full conversion of the second monomer could be detected by IR. Finally, the reaction mixture was poured into a large excess of cold diethyl ether or H₂O (depending on NCA-2) leading to a white polymer after isolation by filtration or centrifugation (3750 rpm, 4 min) and dried under vacuum.

For compound 8, St-PLys-g-PBG-PSar this was the final step of the synthetic procedure.

Yield: 70-90% ¹H NMR (TFA): δ=2.20 (broad m, 1H, CH₂), 2.40 (broad m, 1H, CH₂), 2.71 (broad m, 2H, CH₂), 3.20-3.59 (broad s, 30H, N—CH₃), 4.49-5.01 (broad m, 1H, quiral CH(PBG), 20H, CH₂ (PSar)), 5.32 (broad s, 2H, benzyl CH₂), 7.47 (s, 5H, aryl CH).

For compound 9, St-PLys-g-PBG-PGlu an additional deprotection step of the OtBu group is needed. In brief, the polymer precursor to 9 (St-PLys-g-PBG-P(Otbu)Glu (38.3 g, 0.207 mols) was dissolved in pure TFA (380 mL). The mixture was stirred at r.t. for 3.5 hours. After that time, the solution was poured into diethyl ether to precipitate the product. The precipitate was isolated by filtration or centrifugation (3750 rpm, 4 min) and dried under vacuum yielding an off-white solid.

Yield: 70-90% ¹H NMR (D₂O—HFIP): δ=1.90 (m, 26H, CH₂), 2.20 (m, 26H, CH₂), 4.23 (dd, 12H, quiral CH), 6.97 (broad s, 5H, aryl CH).

For compound 10, St-PLys-g-PHis-PSar, and additional step of the DNP protecting group of histidine with reduced conditions using mercaptoethanol is needed. Briefly, the polymer precursor to 10 is dissolved in DMF (100 mg/mL) and an excess of mercaptoethanol (10 eq) was added to the reaction mixture. The mixture was stirred at r.t. for 16 hours. After that time, the solution was poured into diethyl ether to precipitate the product. The precipitate was isolated by centrifugation (3750 rpm, 4 min) and dried under vacuum. St-PLys-g-PHis-PSar(10) was isolated as a yellow solid.

Yield: 70-90% ¹H NMR (TFA): δ=3.35-3.97 (m, 2H, CH₂ (PHis) 23H, CH₃—NH), 4.95 (s, 15H, CH₂), 7.83 (s, 1H, imidazole), 9.00 (s, 1H, imidazole).

For compound 11, St-PLys-g-PHis-PGlu, both the deprotection of DNP from histidine and OtBu from glutamic acid must be performed in two additional steps. First, the polymer precursor to 10 is dissolved in a mixture DMF/THF 3/1 and an excess of thioethanol (10 eq) was added to the reaction mixture. The mixture was stirred at r.t. for 16 hours. After that time, the solution was poured into diethyl ether to precipitate the product. The precipitate was isolated by centrifugation (3750 rpm, 4 min) and dried under vacuum. St-Plys-g-PHis-PGlu(OtBu) was isolated as a yellow solid.

Secondly, the polymer was dissolved in pure TFA and the mixture was stirred at r.t. for 3.5 hours. After that time, the solution was poured into diethyl ether to precipitate the product. The precipitate was isolated by filtration or centrifugation (3750 rpm, 4 min) and dried under vacuum yielding a yellow solid.

Yield: 70-90% ¹H NMR (D2O-HFIP)

Similarly, and for comparison purposes, the linear analogues to compound 10 (compounds 12) were synthesized using the same procedures but using n-Butylamine as initiator instead of the star-shaped initiator compound 1.

Table 3 Shows different DPs obtained for different Star-Poly(L-Lys)-graft-Poly(L-hydrophobic aa)-block-P(hydrophilic aa), Compounds 8-11. Illustrating a variety of graft copolymers with hydrophobic pockets for hydrophobic molecules encapsulation.

TABLE 3

| | DP | | Graft1 | | Graft2 | G1 | G2 | MW | |
|---|---|---|---|---|---|---|---|---|---|
| C | PLys | Graft1 | Dp^{the} | Graft2 | Dp^{the} | DP^{a} | DP^{a} | kDa^{b} | Ð^{b} |
| 8A | 136 | PBG | 5 | PSar | 50 | n.d. | n.d. | n.d. | n.d. |
| 8B | 136 | PBG | 10 | PSar | 100 | 11 | 108 | 1389 | 1.02 |
| 8C | 136 | PBG | 20 | PSar | 90 | n.d. | n.d. | n.d. | n.d. |
| 8D | 136 | PBG | 30 | PSar | 80 | 42 | 101 | 2247 | 1.07 |
| 8E | 136 | PBG | 60 | PSar | 50 | 77 | 58 | 2904 | 1.06 |
| 9A | 136 | PBG | 10 | PGlu | 100 | 8 | 95 | 1929 | 1.02 |
| 9B | 136 | PBG | 20 | PGlu | 90 | 24 | 81 | 2144 | 1.02 |
| 9C | 136 | PBG | 30 | PGlu | 80 | n.d. | n.d. | n.d. | n.d. |
| 9D | 136 | PBG | 60 | PGlu | 50 | n.d. | n.d. | n.d. | n.d. |
| 10A | 136 | PHis | 10 | PSar | 100 | TBD | | | |
| 10B | 136 | PHis | 30 | PSar | 80 | TBD | | | |
| 10C | 136 | PHis | 60 | PSar | n.d. | | | | |
| 10D | 18 | PHis | 5 | PSar | 10 | 6 | 20 | 42.7 | 1.07 |
| 10E | 18 | PHis | 5 | PSar | 20 | 4.5 | 21 | 55.5 | 1.12 |
| 10F | 33 | PHis | 5 | PSar | 10 | 8 | 21 | 78.3 | 1.06 |
| 11A | 136 | PHis | 10 | PGlu | n.d. | | | | |
| 12A | 11 | PHis | 5 | PSar | 10 | 7 | 16 | 26.1 | 1.04 |
| 12B | 11 | PHis | 5 | PSar | 20 | 5 | 27 | 33.9 | 1.38 |
| 12C | 20 | PHis | 5 | PSar | 10 | 10 | 20 | 47.4 | 1.08 |

^{a}Determined by NMR.
^{b}Determined by SEC.
Mn and DP refer to number average molar mass and degree of polymerization respectively.
Ð represent polydispersity as determined by SEC-MALS software analysis.
N.d.: not determined and in the case of compounds 9C-9D due to solubility problems.
*Note:
the Graft DP relates to the degree of polymerization of each branch.

### Example 1E: Star-Poly((L-Lys)-graft-(Poly(L-Glu))-stat-Poly(Sar), St-(PLys-g-PGlu)-stat-PSar(13)

Compound 13 was synthesized aiming to obtain graft copolymers with the same polymer main chain but a less compact structure. This is achieved by the introduction of unreactive sites in the polymer main chain by statistical co-polymerization of amino acids without functional reactive sites able to initiate a polymerization in a further step. In this particular example, the amino acid NCA chosen to copolymerize with Lys(TFA)NCA is sarcosine NCA as depicted in the scheme 6.

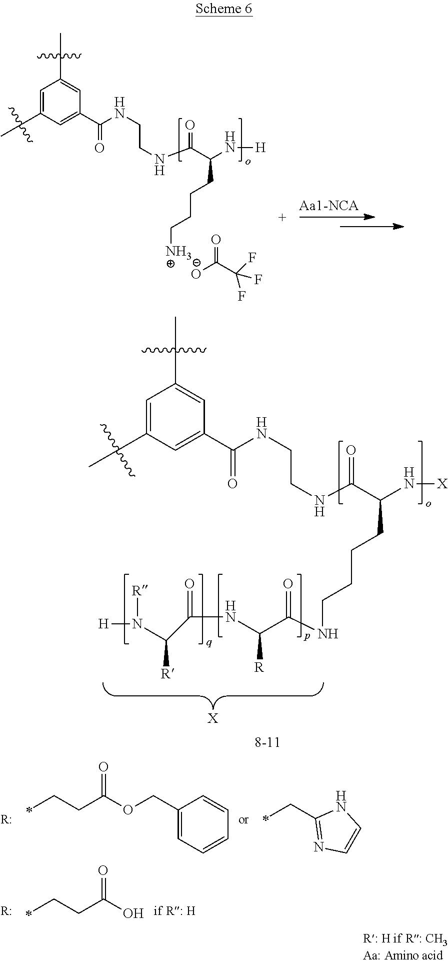

Briefly, the polymer main chain St-PLys-stat-PSar was synthesized similarly to St-PLys but introducing the monomer sarcosine NCA at the same time that Lys(TFA) NCA. After that, the TFA group was deprotected as described for the synthesis of compound 5 St-PLys. Next, the statistical copolymer St-PLys-stat-PSar was used as macroinitator to polymerize the monomer Glu(OBzl) NCA. The benzyl protecting groups of the resulting polymer were removed following the same procedures as described for the synthesis of compound 7.

Table 4 Shows different DPs (degree of polymerization) obtained for St-St-(PLys-g-PGlu)-stat-PSar of formula (13).

TABLE 4

| | DP | Graft | Graft | Graft | Mw | |
|---|---|---|---|---|---|---|
| Compound | PLys/PSAR | polymer | Dp^{the} | DP^{a} | (kDa)^{a} | PDI^{a} |
| 13A | 80/90 | PGlu | 110 | 54 | 553 | 1.05 |

^{a}Determined by SEC.

### Example 1F: Star-Poly(L-Lys)-graft-Poly(Sar)-Poly(Lys-vitE), (14)

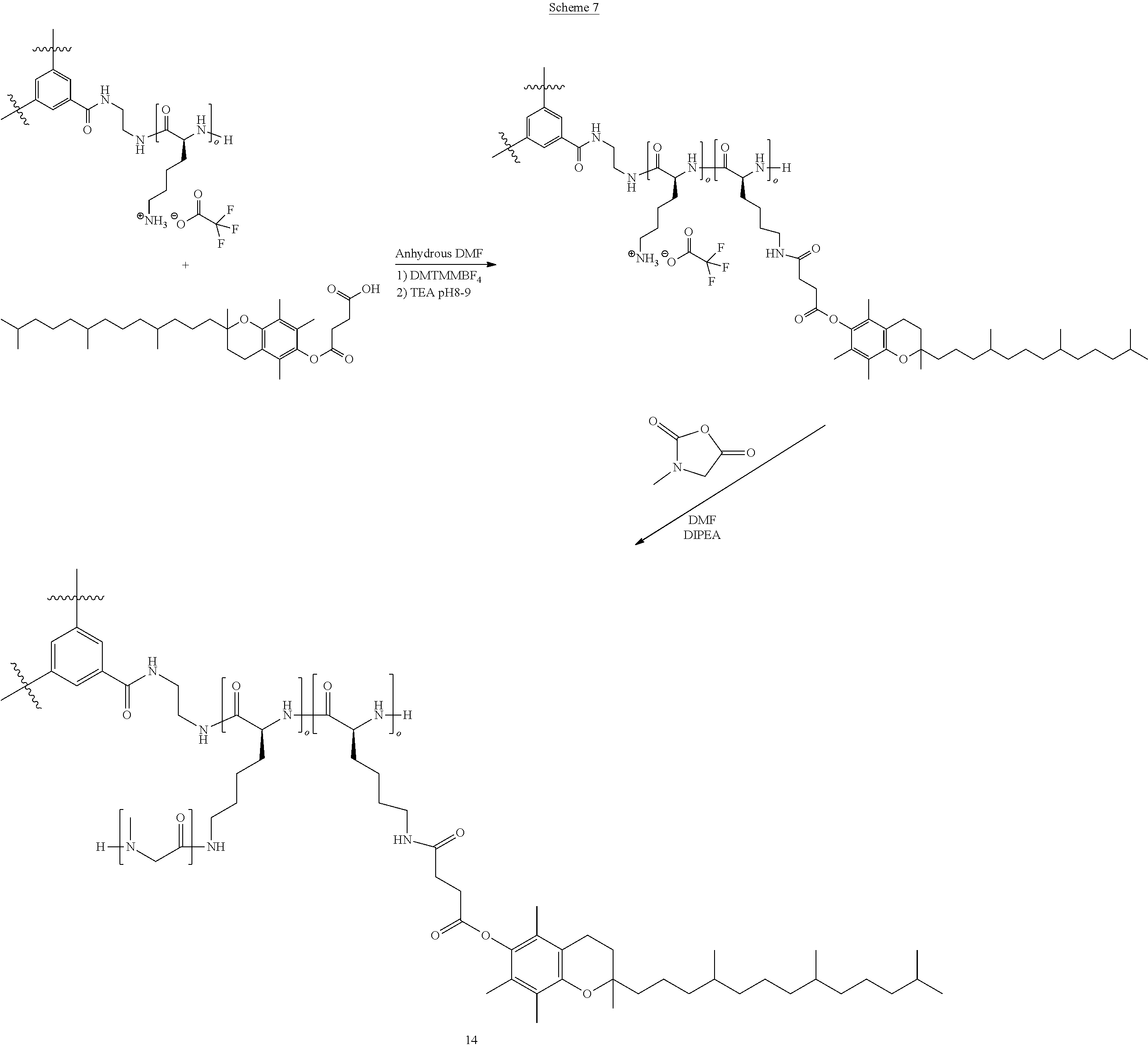

The conjugation of the hydrophobic drug Vitamin E was performed prior to the synthesis of the graft copolymer as shown in Scheme 7. Briefly, St-PLys (compound 5B) (30.0 mg, 0.12 mmol) were dissolved in 0.5 mL of anhydrous DMF. Next DMTMMBF4 (8.1 mg, 0.2 mmol) were added in 0.2 mL of anh. DMF and the reaction mixture was left to proceed for 10-15 min. After that time, Tocopherol succinate (13.2 mg, 0.02 mmol) in anhydrous DMF was added to the reaction mixture and the pH was adjusted to 8-9 with the aid of the addition of 50 UL triethylamine (TEA). The reaction was left to proceed for 24 h at room temperature and under nitrogen atmosphere. After that time, Sar-NCA (228.0 mg, 2.0 mmol) was dissolved in anhydrous DMF (2 mL) and added to the reaction mixture under inert atmosphere. The reaction was left to proceed for 16 hours. Upon completion full conversion of the monomer could be detected by IR. Finally, the mixture was poured in a large excess of diethyl ether and the product was isolated by filtration/centrifugation as a white solid. % of grafting with tocopherol succinate was calculated by ¹H-NMR.

Yield: 70-90% ¹H NMR (MeOD): δ=0.87 (m, 12H, CH₃), 0.94 (t, 3H, CH₃—), 1.02-1.60 (broad m, 32H, CH₂ & CH). 1.98-2.09 (s, 12H, CH₃—), 2.59-3.23 (broad m, 390H, terminal N—CH₃), 3.79-4.66 (broad m, 260H, —CH₂— & —CH₂—).

### Example 1G: Formation of Unimolecular Aggregates from Compounds in Example 1

One of the main characteristics of our graft copolymers is their high molecular weight and the possibility to form unimolecular aggregates with suitable sizes for their application, but not exclusively, in drug delivery. In order to investigate this behavior, we assessed their hydrodynamic size in aqueous solutions by using light scattering techniques. The results obtained for the tested graft copolymers are summarized in Table 5.

Table 5 shows the hydrodynamic size of the graft copolymer unimolecular aggregates formed in solution.

TABLE 5

| Compound | Dh number (nm) | Dh intensity (nm) | PDI |
|---|---|---|---|
| 7A | n.d. | n.d | n.d. |
| 7B | 30 | 45 & 279 | 0.231 |
| 7C | 30* | 62 | 0.243 |
| 7D | 30* | 68 | 0.344 |
| 7E | 29* | 74.76 & 328 | 0.425 |
| 7F | 35 | 66 & 258 | |
| 7G | 40 | 83 | |
| 7H | 49 | n.d. | |
| 8A | 23.10 | 65.50 | 0.240 |
| 8B | 36.17 | 191.3 | 0.253 |
| 8C | Insoluble n.d. | n.d. | |
| 8D | Insoluble n.d. | n.d. | |
| 8E | Insoluble n.d. | n.d. | |
| 9A | 39.59 | 84.90 | 0.226 |
| 9B | 75.07 | 43.83 | 0.134 |
| 9C | 82.38 | 176.4 | 0.178 |
| 9D | 128.9 | 181.0 | 0.103 |
| 10A | 21.34 | 35.83 & 470.9 | 0.325 |
| 10B | 16.22-42.42 | 117.4 | 0.200 |
| 10C (pH = 7) | 28.36 | 156.9-aggregates | 0.414 |
| 10C (pH = 2) | 23.26 | 47.04 | 0.132 |
| 10D (pH = 2) | 5 | 238 & 62 | 0.399 |
| 10D (pH = 11) | 5 | 181 | 0.428 |
| 10E (pH = 2) | 6 | 139 | 0.451 |
| 10E (pH = 11) | 6 | 524 & 65 | 0.520 |
| 12A (pH = 2) | 7 | 117 | 0.266 |
| 12A (pH = 11) | 9 | 423 & 12 | 0.592 |
| 12B (pH = 2) | 7 | 338 | 0.654 |
| 12B (pH = 11) | 8 | 452 | 0.664 |
| 12C (pH = 2) | 9 | 15 | 0.350 |
| 12C (pH = 11) | 8 | 11 | 0.437 |
| 13A | 21.59 | 33.83 & 312.1 | 0.499 |

### Example 2, Conjugation of Small Molecules to Compounds of Formula Ib1

Proof of concept of the conjugation of small molecules has been achieved. As a model molecule, a derivative of the toll-like receptor agonist (TLR7/8) Imiquimod was conjugated to compound 7B as shown in scheme 8 leading to compound 15.

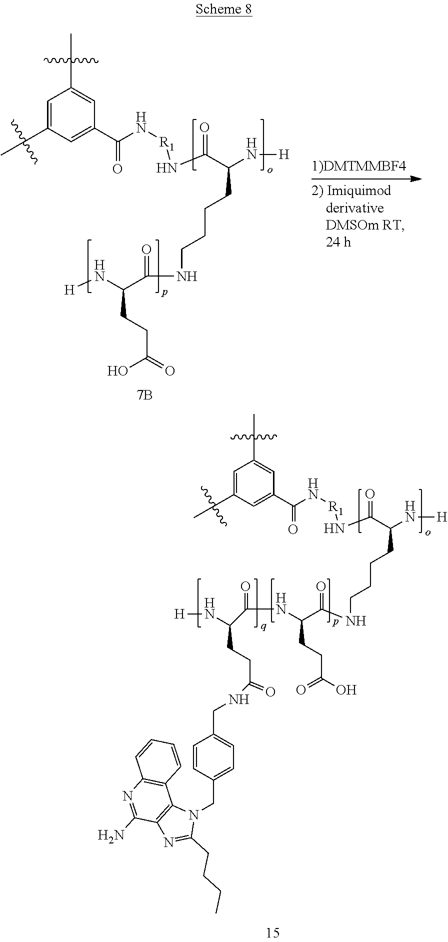

Briefly, the compound 7B was dissolved as acid form in anhydrous DMSO at a concentration of 12-3 mg/mL in a round bottom flask fitted with a stir bar, a stopper and previously purged with N2. Next, DMTMMBF4 (1.5 equivalents of the desired % of modification) was added in anhydrous DMSO to activate the carboxylic acids. The reaction was left to proceed for 10-15 minutes at room temperature. Next, Imiquimod derivative (1.5 equivalents of the desired % of modification) were added and anhydrous DMSO and the pH was adjusted to 8 with the addition of DIPEA base. Final polymer concentration was 10 mg/mL. The reaction was left stirring at room temperature and under nitrogen atmosphere for 20 hours. For purification, the reaction was precipitated in THF and the solid was washed with THF:Et₂O (5:95) three times. For the conversion into the water-soluble sodium salt version of the conjugate, the product was suspended in water and dissolved upon the addition of NaHCO₃ 0.5 M. Next, the compound was de-salted either by Sephadex G25 purification and/or dialysis using Vivaspin MWCO: 30 kDa. A white solid was obtained after water resuspension and lyophilization. The % of TLR 7/8 agonist conjugated was determined by NMR as 3.8 mol % (87.4% yield).

### Example 3. Conjugation of Therapeutic Peptides to Compounds of Formula Ib1

Proof of concept of the conjugation of therapeutic peptides has been achieved. Particularly, two peptide antigens have been conjugated to compound 7B after previous 7B derivatization with pyridyl dithiol ethylamine (PD). Next the two different peptide antigens of major histocompatibility complex class-I and -II (MHC-I and MHC-II), gp100 MHC class I and MHC class II peptides, were conjugated via reversible disulfide bonds (see scheme 9).

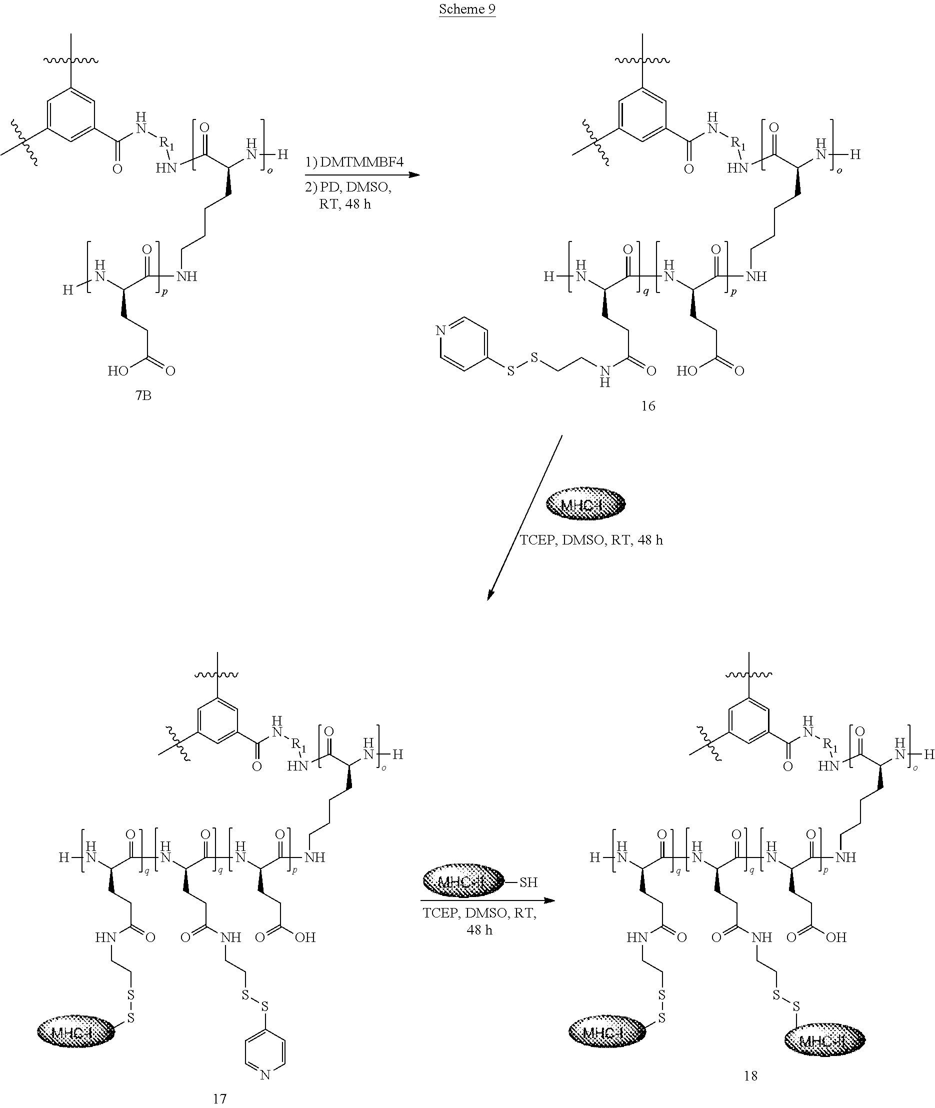

A) Derivatization with pyridyl dithiol ethylamine (PD): Briefly, 900 mg of 7B (6.98 mmol expressed in glutamic acid units, GAU) were added to a 250 mL round bottom flask and purged with N2. Next, 86 mL of DMSO anh. were added and the solid was dissolved after 40 min at RT under agitation. Then, DMTMM.BF4 (148 mg, 0.523 mmol for a 5% GAU derivatization) was added in another 2 mL DMSO anh. After 30 min of reaction, pyridyl dithiol ethylamine (PD) (117 mg, 0.523 mmol for a 5% GAU derivatization) dissolved in 2 mL DMSO anh was added. pH was adjusted to 8 with the addition of DIEA and reaction was left to proceed under stirring for 48 h at RT under anhydrous atmosphere. After that time, reaction was precipitated in 1 L of fresh THF under agitation and cooled down to 4° C. for 16 hours to favor precipitation. Then, the solid was collected by centrifugation, washed with THF and THF:Et₂O (5:95) and lyophilization yielded a white solid of compound 16. Identity, purity and conjugation efficiency was determined by ¹H-NMR in D₂O with a tiny addition of sodium bicarbonate. Yield: 90% (1050 mg), conjugation efficiency: 70% (3.5% mol GAU derivatization with PD).
B) Peptide conjugation to PD-modified graft copolymers (compound 17 and 18): Briefly, for MHC-I peptide conjugation the PD-modified graft copolymers (350 mg, 2.57 mmol GAU) were dissolved in 35 mL of DMSO anh. Next, 59.72 mg of MHC-I peptide, (0.0495 mmol, 0.019 eq for 1.9% modification, 1207 g/mol) were dissolved in 5 mL of anhydrous DMSO followed by the addition of 0.15 eq of Tris(2-carboxyethyl) phosphine hydrochloride, TCEP (0.722, 0.0029 mmol). The reaction was left to proceed for 48 hours at RT, under stirring and N2 atmosphere. After that time, the compound was precipitated in cold THF with a ratio 1:20. The precipitate was isolated by centrifugation and was washed with THF. A white solid was finally obtained by lyophilization. This solid was transformed into the sodium salt version of the product by addition of NaHCO₃ and subsequent desalting dialysis using 30 kDa Vivaspin. Peptide content was determined as 6.07% wt by amino acid analysis. Yield: 40% Conjugation efficiency: 49% (0.86% mol MHC-1 conjugated).

In another example, gp100 MHC-I and MHC-II peptides were conjugated in tandem in the same polymeric backbone (compound 18). Briefly, after 48 hours reaction with MHC-I peptide as described above, instead of precipitating the gp100 MCH-I conjugate, the reaction was left to proceed for further 48 hours after the addition of gp100 MHC-II peptide (0.019 eq for 1.9% modification, 2208 g/mol) dissolved in 5 mL of anhydrous DMSO and of 0.15 eq of TCEP (0.722, 0.0029 mmol). After that time, the compound was precipitated in cold THF with a ratio 1:20. The precipitate was isolated by centrifugation and was washed with THF. A white solid was finally obtained by lyophilization. This solid was transformed into the sodium salt version of the product by addition of NaHCO₃ and subsequent desalting dialysis using 30 kDa Vivaspin. Peptide content was determined as 4.11% wt for MHC-I peptide and 4.99% wt for MHC-II peptide by amino acid analysis. Yield: 60% Conjugation efficiency: 36%.

### Example 4. Encapsulation of Drugs in Compounds of Formula Ib1

Compounds 8-12, depicted in the example 1D and 1E were designed to hold hydrophobic pocket for drug encapsulation due to their amphiphilic nature and the presence of a highly hydrophobic block of polyamino acids (i.e. PBLG or PHis).

In a first attempt and to characterize the system, Nile Red as a model hydrophobic drug with log P: 2.98 was chosen.

In the particular case of compounds 9, St-PLys-g-PBG-PGlu, the capacity to incorporate the hydrophobic molecule of Nile Red within the structure demonstrated the presence of hydrophobic pockets. Nile Red is commonly used as lipophillic stain. In hydrophilic environments, Nile Red will hardly fluoresce but when encapsulated/confined in hydrophobic environments it can be intensely fluorescent, with varying colours from deep red (for polar membrane-like environments) to strong yellow-gold emission (for neutral). The existence of the hydrophobic pocket and the response of the hydrophobic/hydrophilic balance of the polymer to changes in pH is determined by fluorescence Nile Red assays. This test is based on the pronounced solvatochromic effect and increased fluorescence that this molecule emits in the presence of the hydrophobic microenvironments as mentioned. To do so, a growing amount of 5 mM Nile Red in ethanol solution (from 10 to 120 μM final concentration, see table below) was added to a 10 mg/mL solution of the polymer in water. After mixing the samples for 24 hours at room temperature, the samples were centrifuged at 13300 for 2 minutes. Next, the absorbance at 560 nm was measured by triplicate in a 96 dark well plate using a plate reader.

Table 6. Shows the maximum Nile Red encapsulated in ppms at different concentrations in compounds 9.

TABLE 6

| | Conc. | Blank Samples | | Samples | | | | | |
|---|---|---|---|---|---|---|---|---|---|
| | Nile | uL Nile | | μL Nile | μL | Measurement at 560 nm | | | Max. Nile Red |
| | Red | Red 5 | μL | Red 5 | Polymer | Abs | Abs | | Encapsulated |
| # | (uM) | mM | H2O | mM | 10 mx | Blank | Samples | Abs | (ppm) |
| 1 | 10 | 2 | 998 | 2 | 998 | 0.057 | 0.095 | 0.038 | 3.1837 |
| 2 | 20 | 4 | 996 | 4 | 996 | 0.057 | 0.173 | 0.116 | 6.3674 |
| 3 | 40 | 8 | 992 | 8 | 992 | 0.083 | 0.225 | 0.142 | 12.7348 |
| 4 | 80 | 16 | 984 | 16 | 984 | 0.087 | 0.313 | 0.226 | 25.4696 |
| 5 | 120 | 24 | 976 | 24 | 976 | 0.075 | 0.314 | 0.240 | 38.2044 |

In the particular case of compounds 10 and 12 where a pH sensitive hydrophobic block of histidine is present, a potentiometric titration was performed in order to find the hydrophobic range of drug encapsulation prior to Nile red assays.

Potentiometric titration: The pKₐ of a cationic polymer is determined by acid-base titration, measuring the pH of the solution throughout the process. The pKₐ is then obtained from the titration graph. To carry out the measurement, a 1 mg/mL solution of the cationic polymer is prepared in Milli-Q water and a known quantity of HCl 0.1M is added until the pH of the solution is around 2. At this point, the titration is performed with NaOH 0.2 M using an automatic Methrom 916 ti-touch potentiometer with a Dosin 800 dispenser. The titration speed is set to 0.1 mL/min with a signal drift of 50 mV/min. The titration is complete when the pH reaches 12. The relationship between pH and the protonation degree of polycations was calculated from the obtained titration curves. Furthermore, pK=pH+log [α/(1−α)] values were plotted against 1−α, where K is the effective dissociation constant.

Table 7 shows the pKₐ values obtained for the different compounds.

TABLE 7

| Compound | Description | pka |
|---|---|---|
| 10D | St-PLys(18)-Hist(5)-Sar(10) | 5.427 |
| 10E | St-PLys(18)-Hist(5)-Sar(20) | 5.43 |
| 10F | St-PLys(33)-Hist(5)-Sar(10) | 5.472 |
| 12A | nBu-PLys(11)-Hist(5)-Sar(10) | 5.197 |
| 12B | nBu-PLys(11)-Hist(5)-Sar(20) | 5.383 |
| 12C | nBu-PLys(20)-Hist(5)-Sar(10) | 5.201 |

With these molecules, the Nile Red test was modified. The test consists of preparing two solutions of Nile Red in Mili-Q water at pH 2 ([Nile Red]=30 μM). with and without polymer. Successive volumes of 1 M NaOH (10 μL) were added to these solutions until a final pH of 11 was reached. Nile Red is insoluble in polar solvents such as water, so do not show fluorescence. After adding NaOH, and increasing the pH of the medium in the presence of a hydrophobic environment (sample containing polymer), it is observed how the color of the solution is highly violet and fluorescence is observed, which shows that the polymer has been capable of encapsulating the hydrophobic molecule allowing it to be soluble in aqueous media. In the solution that does not contain polymer, no changes are observed, there is no fluorescence signal.

Apart from Nile Red, other exemplary hydrophobic dyes such as DIL and molecules/drugs such as Vitamin E were also successfully encapsulated using the same procedure.

DIL also known as DilC18(3), is a fluorescent lipophilic cationic indocarbocyanine dye (c Log P: 22.539) which has an absorption maximum at 550 nm and an emission maximum 565 nm. DIL encapsulation was demonstrated by Size Exclusion Chromatography (SEC) measurements and visual observations. For this, a blank of DIL in PBS buffer was prepared, and after filtration of the sample, a colorless solution was observed indicating that the DIL dye was kept insoluble in PBS in the filter. However, when the DIL dye was encapsulated within the graft copolymer micelles core and this solution was filtered off, the colorimetric properties of DIL could be visually appreciated as the solution maintained the pink color. SEC measurements using a Diode-array detector, DAD detector with λ_{abs}=550 nm confirmed the absorbance of encapsulated DIL only, when compared with the filtered blank and the polymer alone.

Similarly, the partial encapsulation of Vit E within these polymers was demonstrated. Vitamin E partial encapsulation was assessed by visual observations (similarly as for DIL, before and after aqueous solutions filtration) and by turbidimetry assays monitoring the absorbance at high A attributed only to the turbidity of the sample. The turbidity decrease observed indicated vit E encapsulation.

### Example 5. Biological Evaluation: Biodistribution Profiles of Compounds of Formula Ib1

Biodistribution experiment and fluorescence quantification. Biodistribution experiments were carried out using male Balb/c mice of 5-7 weeks from Envigo. Animals were housed on a 12-hour light and 12-hour dark cycle. Water and food was provided ad libitum during the whole experiment in all cases, and general aspect, behavior and body weight were evaluated daily to ensure animal wellness. All animal protocols were approved by the Institutional Animal Care and Use Committee at the Centro de Investigación Príncipe Felipe (CIPF, Valencia, Spain). For biodistribution study, six mice at each time point were used of 25+4.5 g of body weight). The labeled polymer was administered intravenously through the tail vein at a dose of 1 mg/Kg Cy5.5 eq. in serum saline. Mice were euthanized at 0 h, 1 h, 4 h, 24 h, post administration. Cy5.5 was injected to other 3 mice in the same way at 1 mg/Kg for 4 hours and 3 mice were used as controls with PBS injection for basal fluorescence. Blood, kidney, liver, spleen, heart, lungs, axillary and inguinal lymph nodes were harvested after flushing with 10 mL PBS and their fluorescence was measured in IVIS® Spectrum (PerkinElmer, Waltham, MA, USA). Then, organs were weighted and stored at −80° C. for subsequent image acquisition by IVIS Spectrum and fluorescence quantification. For image acquisition by IVIS Spectrum the settings used were (Exc 640/Em 700, Bining Medium, F/Stop 2, Exp 0.5 sec, FOV 13.2 (C)). After that, images were analysed using Living Image 4.5.4 software. Region of interest (ROI) was drawn automatically for each organ and manually for basal organ with a circle ROI. Lymph nodes were analysed separately. The Total Radiance Efficiency ((p/s)/(μW/cm²) of interest organs was taken and the basal values were subtracted. Then, Total Radiance Efficiency was normalized according to the organ weight.

Results show a differential biodistribution profile for the two different graft copolymers depending on their physico-chemical characteristics. The graftcopolymer compound 7B shows a renal excretion profile with accumulation in major organs such as liver and spleen, and a specific targeting of inguinal and axillary lymph nodes (ILN and ALN respectively). This selective accumulation makes this type of compounds excellent candidates for lymphotropic delivery. In the case of compound 9A which includes the incorporation of hydrophobic pockets, the accumulation in major organs such as liver and spleen is increased with respect to the one found for 7B without a clear renal excretion profile. In this case, the introduction of hydrophobic moieties changes completely the specific targeting towards the lymph nodes, which does not occur.

### Example 6. Biological Evaluation in a Primary Model of B16F10 Melanoma Mice Model

Immune-modulatory potential of graft copolymers St-PLys-g-PGlu(compound 7B). High efficacy and High response rate with a Safe Profile: Preliminary data obtained. The compounds obtained in example 3, where gp100 MHC class I and MHC class II peptides have been bound to St-Plys-g-PGlu (aka, St-Br) as single conjugates (St-Br-gp100 MHCI, St-Br-gp100 MHCII) or in combination (St-Br-gp100 MHCI_gp100 MHCII)) were evaluated in terms of their anti-tumor effect in a primary model of B16F10 melanoma, the most aggressive type of skin cancer.

Moreover, a ligand of the Toll-like receptors 7/8 has also been conjugated to St-Br ([St-Br-TLR7/8], compound described in Example 2) to deliver adjuvant-like signals to antigen-presenting cells (APCs) and thereby improve the antigen presentation towards an extensive T-cell activation and expansion (ref: Frega, G. et al. Trial Watch: experimental TLR7/TLR8 agonists for oncological indications. Oncoimmunology 9, 1796002 (2020)). Thus, the anti-tumor immune-mediated effect induced by the St-Br conjugated with a TLR7/8 agonist (was evaluated alone and in combination with the St-Br conjugate delivering both gp100 MHC I and MHC II peptides, and compared with the response induced by this dual conjugate mixed with Toll-like Receptor (TLR) 9 (CpG) and TLR3 agonists. The average tumor volume of mice treated with [St-Br-gp100 MHCI-gp100 MHCII] and TLR inductors, CpG and Poly I:C, were significantly lower compared to PBS treated group, 21 post-tumor inoculation (P=0.0335). None of the St-Br conjugates led to significant variations in mouse body weight, which indicates the tolerability and safety of these carriers. Mice were sacrificed on day 22 post-tumor inoculation and tumors were collected and later processed for FACS analysis.

The dual nanoconjugate [St-Br-gp100 MHCI-gp100 MHCII] mixed with CpG and Poly I:C significantly induced the infiltration of CD3⁺, and CD4⁺ T cells, and reduced the percentage of PD-1⁺ expressing CD3⁺ cells. Significant higher levels of antigen-specific intracellular IFN-γ⁺ and TNF-α⁺ in tumor-infiltrating cytotoxic CD8⁺ T lymphocytes were also observed. The higher infiltration of T lymphocytes correlates with the stronger tumor growth inhibition induced by [St-Br-gp100 MHCI_St-Br-gp100 MHCII] with CpG and Poly I:C.

[St-Br-gp100MHCI-gp100MHCII] combined with CpG and Poly I:C promoted the infiltration of myeloid DC2, while myeloid DC1 cells, which are correlated to stronger cytotoxic immune responses within TME, were found at higher levels in the TME of animals treated with the [St-Br-TLR7/8] alone or combined with [St-Br-gp100MHCI] or [St-Br-gp100MHCI-gp100MHCII].

The immune-mediated effect of these multivalent nanovaccines was also evaluated in a humanized patient-derived orthotopic melanoma model. To confirm the humanization process, the presence of human lymphocytes (human CD45⁺ cells) in murine blood was assessed at days 7, 14 and 21 post-tumor cell inoculation. Human CD45⁺ cells in the total lymphocyte population increased along the time, from 13.3% on day 7 to 71.6% on day 21 post-tumor cell inoculation. At day 17 (endpoint day), 97.1% and 79.1% of CD45⁺ cells in the blood and in the spleen, respectively, were human, which confirms the successful development of this melanoma patient-derived mouse model. Mice were sacrificed on day 17 post-treatment initiation and tumors and spleens were collected and later processed for FACS analysis.

Humanized mice were treated with the conjugated branched polypeptides [St-Br-gp100MHCI-gp100MHCII] alone or combined with PD-1 antibody (αPD-1) Nivolumab. From day 15 post-treatment, both treatments significantly decreased the tumor growth compared to PBS-treated group (P=0.0081 and P=0.0414, for [St-Br-gp100MHCI-gp100MHCII]+TLRI alone or combined with αPD-1, respectively).

However, no significant differences were observed among them. At day 17 after treatment initiation, the individual tumor weight of mice was significantly reduced in the animals immunized with [St-Br-gp100MHCI-gp100 MHCII]+TLRI alone (P=0.0070), or in combination with αPD-1 (P=0.0381). No significant differences were observed in the weight of mice over time indicating negligible systemic toxicity.

[St-Br-gp100MHCI-gp100MHCII] combined with αPD-1 led to an extensive tumor infiltration of T lymphocytes (P<0.0001). Additionally, CD8⁺ T cells were highly increased in spleen of [St-Br-gp100MHCI-gp100MHCII] combined with αPD-1 (P<0.0001). Moreover, PD-1 expression was significantly decreased in CD3⁺ and CD8⁺ T cells quantified in the spleen, compared to those presented by animals treated with the [St-Br-gp100MHCI-gp100MHCII] alone (P<0.0001, for both treatments).

Overall, these findings demonstrated the potential of St-Br conjugated to MHCI and MHCII restricted antigen and/or TLR ligands as cancer vaccine to modulate the melanoma-immune cells crosstalk within microenvironment. Moreover, results obtained following the administration of this dual nano-conjugate to the melanoma patient-derived xenograft (PDX) model validate the potential clinical application of these St-Br conjugates as multivalent cancer vaccines.

### Example 7. Preparation of Compounds of Formula (I)—Glu Bound to Branches with Different Amino Acid Structures (Ib2)

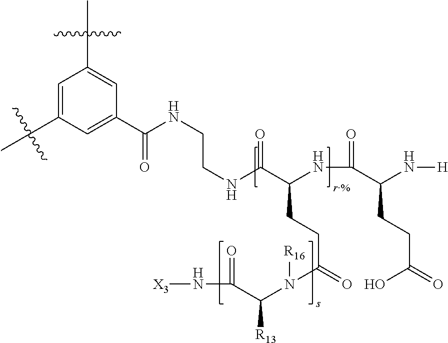

In relation to formula (Ia) of the first aspect herein-formula (Ib2) above is an example, wherein the integer “r” is ≠0 and β is an integer of 1—i.e. relating to use of

Glu to bind branches with different amino acid structures.

Compounds 19-20 of formula (Ib2) were synthesized according to the next synthetic scheme 10.

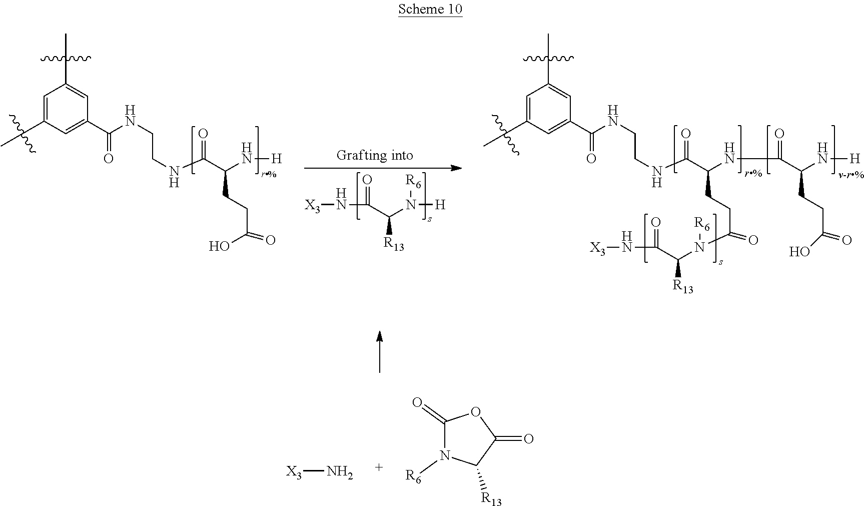

In brief, the precursor St-PGlu was synthesized as previously described for compound 4, but using γ-Benzyl L-glutamate NCA. In a second step, the benzyl protecting group is deprotected under acidic conditions. Briefly, the polymer is dissolved in pure TFA and the mixture was left to react at r.t. for 3 hours. After that time, the solution was poured into diethyl ether to precipitate the product. The product St-PGlu was isolated by centrifugation (3750 rpm, 4 min) and dried under vacuum as a white solid.

In a next step, the polymer St-PGlu(20.0 mg, 0.16 mmol PGlu units) was dissolved in anhydrous DMF (2 mL). Next, 0.4 equivalents of the activating agent DMTMM were added to the reaction mixture and it was left to proceed for 10-15 min at room temperature in nitrogen atmosphere. After that time, the desired equivalents of pre-synthesized polyamino acid chains (i.e. polysarcosine (352.0 mg, 0.06 mmol), polylysine-thioglycerated, for a certain percentage of grafting were added to the reaction mixture dissolved in 4 mL of anh. DMF. The pH was adjusted to 8-9 with TEA (150 μL) and the reaction was left to proceed for 48 hours at room temperature and under nitrogen atmosphere. After that time, the solution was poured into diethyl ether to precipitate the product. The product was isolated by centrifugation (3750 rpm, 4 min) and dried under vacuum as a white solid.

The degree of grafting and grafting efficiency was estimated by NMR. Yield: 70-90% ¹H NMR (D₂O): δ=1.21 (m, 6H, CH₃), 2.05 (m, 5H, CH₂), 2.35 (m, 5H, CH₂), 2.83-3.30 (m, 290H, N—CH₃), 4.06-4.64 (m, 190H, quiral CH, CH₂). Grafting %=27.66%. Grafting efficiency %=69.15%.

Table 8 shows the two R₁₃ and R₆ examples.

TABLE 8

| Graft polymer | R13 | R6 |
|---|---|---|
| PSar (poly-sarcosine) | 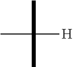 | 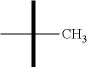 |
| PLys-TG (poly-lysine-thioglycerated) | 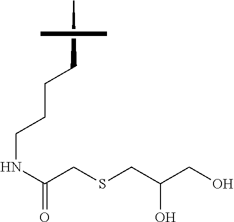 | 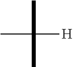 |

As an example, the general procedure to obtain the previously polymerized graft chains is described for PSar: Sarcosine-NCA (1675 mg, 14.59 mmol) was added to a Schlenk tube fitted with a stirring bar, a stopper, then purged with 3 cycles of vacuum/N2, and dissolved in 6 mL of anhydrous DMF. Then, isopropylamine was dissolved in DMF (2 mL) and added to the reaction mixture. The mixture was stirred at 10° C. for 16 hours. Upon completion, the reaction mixture became clear. Full conversion of the monomer could be detected by IR. Then, the reaction mixture was poured into diethyl ether to precipitate the product. The precipitate was isolated by centrifugation (3750 rpm, 4 min) and dried under vacuum. The final product was isolated as a white solid.

Yield: 80%. ¹H NMR (D₂O): δ=1.08 (d, J=6.7 Hz, CH₃), 2.73-3.12 (m, CH₃), 3.78-4.56 (m, CH₂). MW by SEC: 1222 Da (1.104)

Table 9 shows the main characterization of compounds 19 and 20.

TABLE 9

| | | Main | | | % | | | | |
|---|---|---|---|---|---|---|---|---|---|
| | Main | chain | Graft | Graft | Grafting | % | Mw^{a} | Mw^{b} | |
| C | chain | DP | chain | DP | theo | Grafting^{a} | (KDa) | (KDa) | Ð^{a} |
| 19A | St-PGlu | 150 | Psar | 80 | 5 | 2.34 | 37.9 | n.d. | n.d. |
| 19B | St-PGlu | 150 | Psar | 80 | 10 | 9.16 | 97.1 | 96.4 | 1.07 |
| 19C | St-PGlu | 150 | Psar | 80 | 40 | 27.66 | 257.9 | n.d. | n.d. |
| 20A | St-PGlu | 150 | PLysTG | 25 | | | | | |

^{a}Data obtained by ¹H-NMR in D₂O.
^{b}Data obtained by SEC in MeOH•GuHCl.

### Example 8. Synthesis of Compounds of Formula (Ib3)

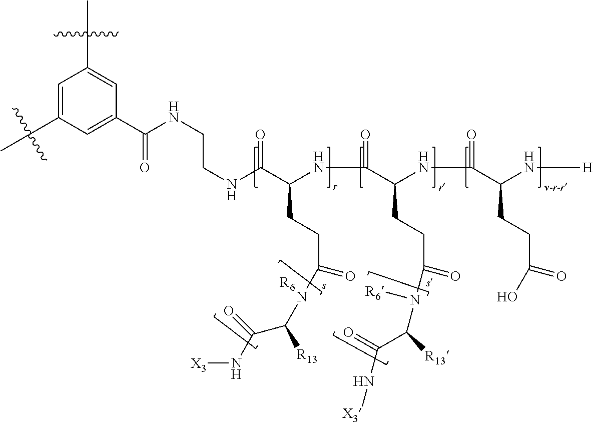

Similarly to compounds in Example 7, compounds 21 were synthesized according to the next synthetic scheme 11.

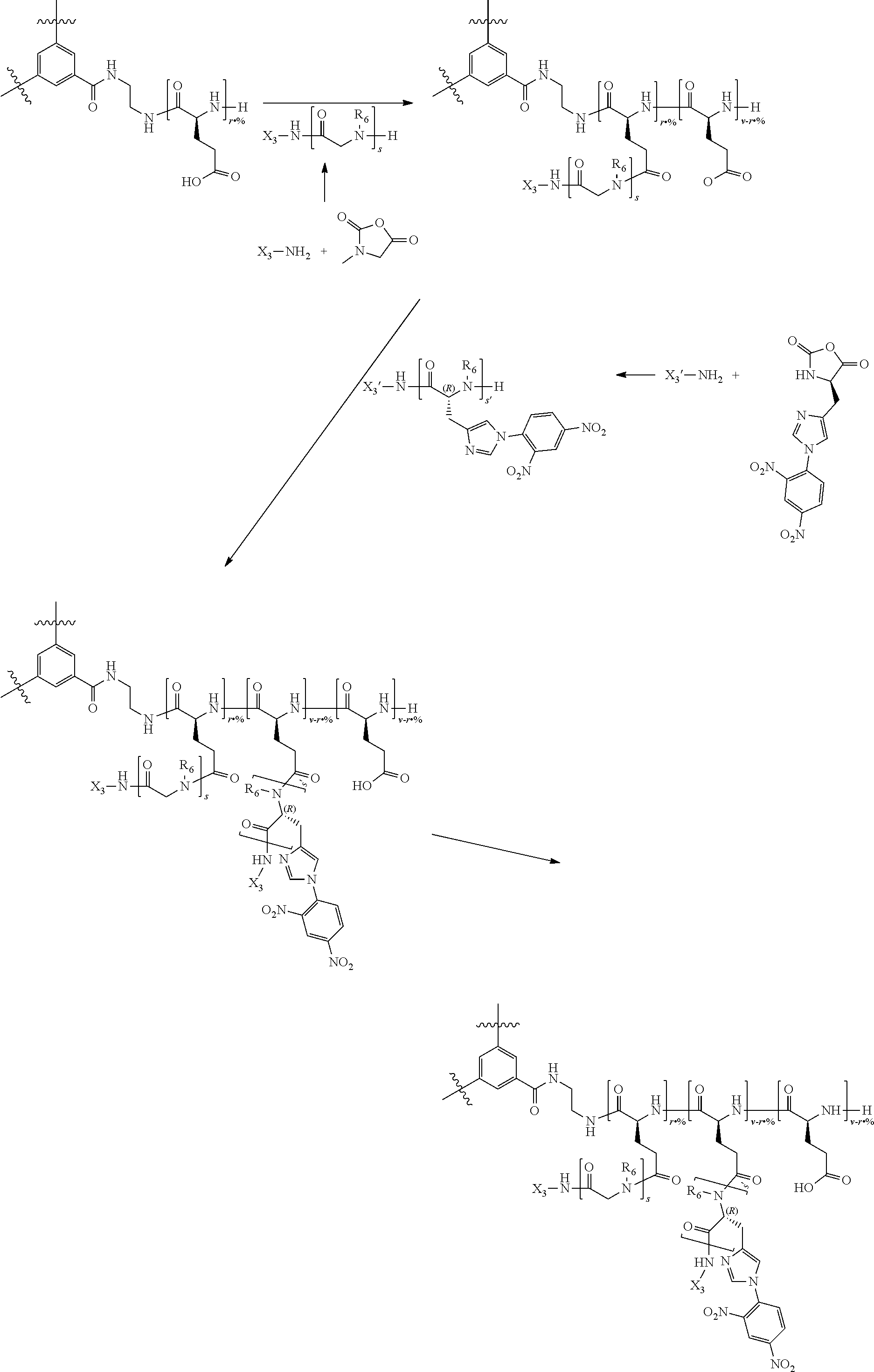

In brief, the precursor star-PGlu was synthesized as previously described using γ-Benzyl L-glutamate NCA. In a second step, the benzyl protecting group is deprotected under basic conditions. Briefly, the polymer is dissolved in THF (100 mg/mL) and NaOH (1.5 eq) was added. The mixture was stirred at 4° C. for 16 h. Upon completion, the reaction mixture was poured into tert-butylmethyl ether:acetonitrile (3:1) to precipitate the product. The precipitate was isolated by filtration and dried under vacuum. Homopolymer was isolated as a white solid. Yield: 70-90%. ¹H NMR (D₂O): δ=1.69-2.60 (m, 2H, CH₂), 3.57 (m, 2H, CH₂); 3.97-4.39 (m, 1H, CH), 8.32 (s, 1H, aryl CH).

In a similar way to the polymerization of PBG, the synthesis of iPrPSar(16) and nBuPHis(DNP) (5) was carried out. As an example, the synthesis of nBuPHis(DNP) (5) is described:

Scheme 12 shows the general procedure for the polymerization of PHis(DNP).

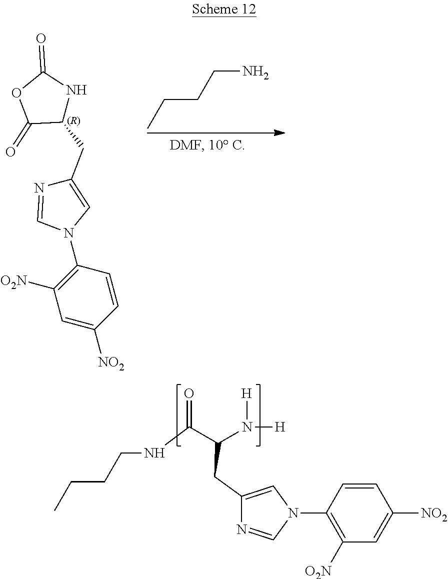

Briefly, L-His(DNP)NCA (2000 mg, 5.76 mmol) was added to a Schlenk tube fitted with a stirring bar, a stopper, then purged with 3 cycles of vacuum/N2, and dissolved in 8 mL of anhydrous DMF. Then, butylamine was dissolved in DMF (2 mL) and added to the reaction mixture. The mixture was stirred at 10° C. for 16 hours. Upon completion, the reaction mixture became clear. Full conversion of the monomer could be detected by IR. Then, the reaction mixture was poured into diethyl ether to precipitate the product. The precipitate was isolated by centrifugation (3750 rpm, 4 min) and dried under vacuum. The final product was isolated as a brown solid. Yield: 80%. ¹H NMR (DMSO): δ=0.81 (t, J=7.1 Hz, CH₃), 1.06-1.40 (m, CH₂), 3.37 (m, CH₂), 4.48 (m, CH), 7.20 (m, CH), 7.82-8.08 (m, CH), 8.22-8.46 (m, NH amide), 8.64 (brs, CH), 8.90 (brs, CH).

Next, the both polymers (iPrPSar and nBuHis(DNP)) were sequentially conjugated to the main PGlu chain by activating with DMTMMBF₄ as follows. St-PGlu(0.1 g) was added to a two-necked round bottom flask fitted with a stirring bar and a stopper, then purged with 3 cycles of vacuum/N2, and dissolved in 1 mL of DMF. Then, DMTMMBF4 (4 eq, 0.366 g) was added to the reaction mixture and stirred for 30 minutes at room temperature. After this time, the polymer iPrPSar dissolved in 0.5 mL of DMF was added. The mixture was stirred at room temperature for 16 hours. The reaction mixture was poured into diethyl ether to precipitate the product. Precipitate was isolated by centrifugation (3750 rpm, 4 min) and dried under vacuum. The final product was isolated as a white solid. Yield: 80%.

For the coupling of the polyhistidine polymer, the procedure described above was followed. The final product was isolated as a yellowish solid. Yield: 80%.

The final step consists of the DNP protecting group of histidine under reduced conditions is needed. Briefly, the polymer precursor to 10 is dissolved in DMF (100 mg/ml) and mercaptoethanol (10 eq) was added to the reaction mixture. The mixture was stirred at room temperature for 16 hours. After that time, the solution was poured into diethyl ether to precipitate the product. The precipitate was isolated by centrifugation (3750 rpm, 4 min) and dried under vacuum. St-PGlu-graft into-PSar-graft-into-PHis was isolated as a yellowish solid.

Yield: 70-90%

Table 10 shows the composition of these graft copolymers and main characterization.

TABLE 10

| | Main | DPmain | | DP | | | DP | | MW^{b} | |
|---|---|---|---|---|---|---|---|---|---|---|
| C | chain | chain^{a} | Graft1 | Graft1^{a} | % Graft1^{a} | Graft2 | Graft2^{a} | % Graft2^{a} | kDa | Ð ^{b} |
| 21A | St- | 12 | PSar | 16 | 63 | PHis | 5 | 25 | 12.2 | 1.14 |
| | PGlu | | | | | | | | | |
| 21B | St- | 26 | PSar | 16 | 44 | PHis | 5 | 30 | 16.8 | 1.10 |
| | PGlu | | | | | | | | | |

^{a}Data obtained by ¹H-NMR.
^{b}Data obtained by GPC in MeOH/Guanidium

### Example 9: Formation of Unimolecular Aggregates from Compounds of Formula Ib2

One of the main characteristics of our graft copolymers is their high molecular weight and the possibility to form unimolecular aggregates with suitable sizes for their application, but not exclusively, in drug delivery. In order to investigate this behavior, we assessed their hydrodynamic size in aqueous solutions by using light scattering techniques. The results obtained for the tested graft copolymers are summarized in table 11.

Table 11 shows the hydrodynamic size of the graft copolymer unimolecular aggregates formed in solution.

TABLE 11

| Compound | Dh number (nm) | Dh intensity (nm) | PDI |
|---|---|---|---|
| 19A | 12.8 | 30.75 & 316 | 0.724 |
| 19B | 12.7 | 17.68 & 258 | 0.894 |
| 19C | 18.9 | 35.14 & 274 | 0.408 |

### Example 10. Conjugation of Drugs to Compounds of Formula (Ib2)

The conjugation of hydrophobic drugs to compounds 19, St-PGlu-g-Psar, have been successfully performed to get compound 22 below. In this particular example, glycine-vitamin E was conjugated to St-PGlu-g-Psar (10%) following the conditions described in scheme 13.

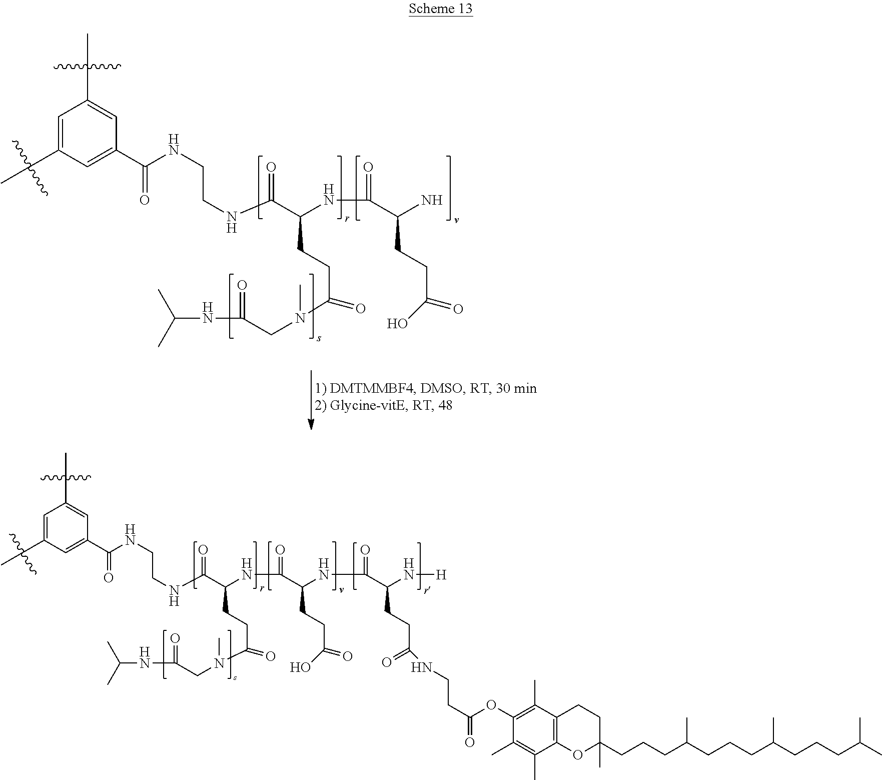

Briefly, 50 mg of St-PGlu-g-PSar(10%) in sodium salt form (0.058 mmol, 1 eq) was dissolved in DMSO anhydrous (2 mL) in a 250 mL round bottom flask fitted with a stir bar and a septum stopper. After that, 3.92 mg (0.012 mmol, 0.2 eq) of DMTMMBF4 was added in another 1 mL of DMSO. In this particular case, the amount of DMTMMBF4 added corresponded to a % of glutamic acid units modification of 20%. The reaction was left to proceed for 30 minutes under vigorous stirring and inert atmosphere. Next 7.15 mg (0.015 mmol, 0.25 eq) of glycine-modified vitamin E was added to the reaction mixture in 1 mL of DMSO. The reaction was left stirring for another 48 hours. After that time, the product was poured in a large excess of diethyl ether. A white solid was isolated by centrifugation and freeze-drying. Yield: 36 mg; conjugation efficiency, 65%; % of Vit E incorporated: 13% determined by ¹H-NMR.

### Example 11. Encapsulation of Hydrophobic Molecules in Compounds of Formula Ib3

Compounds 21, depicted in the example 8 were designed to hold hydrophobic pocket for drug encapsulation due their amphiphilic nature and the presence of a pH sensitive and highly hydrophobic block of PHis.

In a first attempt and to characterize the system, Nile Red as a model hydrophobic drug with log P: 2.98 was chosen. A pH titration was also performed in order to find the hydrophobic range of drug encapsulation. The procedure followed was as described for compounds 10-12. The pka for compound 21A was found to be 6.45 and the Nile Red assay showed the transition from hydrophilic to hydrophobic environment upon the increase in pH.

### Example 12. Preparation of New Compounds of Formula (I)—Lys Bound to Branches with Different Amino Acid Structures

Compounds 22A-G are new examples based in the previous compounds described in the section Example 1. Namely, compounds 22A, 22C and 22D are synthesized following the procedure described in Example 1C, and compounds 22B, 22E-G are synthesized following the procedure described in Example 1E, by changing the degree of polymerization and structure. The design of these polymers aims to vary structural parameters such as morphology, compactness, and lower ionic charge.

TABLE 12

| | | | | Dh | Dh | |
|---|---|---|---|---|---|---|
| Com- | Compound | MW | | number | intensity | |
| pound | name | (kDa)^{a} | PDI^{a} | (nm) | (nm) | PDI |
| 22A | StPLys(135)-g- | 1183 | 1.13 | 47.21& | 31.55 | 0.485 |
| | PGA(68) | | | 284.80 | | |
| 22B | St(PLys(60)-b- | 386 | 1.06 | 37.05 | 20.00 | 0.406 |
| | PSar(70))-g- | | | & 456.80 | | |
| | PGA(50) | | | | | |
| 22C | StPLys(71)-g- | 762 | 1.05 | 33.91& | 22.05 | 0.404 |
| | PGA(83) | | | 182.70 | | |
| 22D | StPLys(1314)- | 2066 | 1.09 | 132.20 | 25.6 | 0.393 |
| | g-PGA(12) | | | | | |
| 22E | StPLys(71)-g- | 366 | 1.01 | 28.57& | 19.74 | 0.408 |
| | (PGA(22)-stat- | | | 391.50 | | |
| | PSar(34)) | | | | | |
| 22F | St(PLys(60)-b- | 436 | 1.03 | 35.87& | 24.22 | 0.781 |
| | PSar(70))-g- | | | 333.00 | | |
| | PGA(57) | | | | | |
| 22G | St(PLys(80)-b- | 869 | 1.01 | 49.67& | 31.68 | 0.467 |
| | PSar(300))-g- | | | 382.00 | | |
| | PGA(85) | | | | | |

^{a}Data obtained by SEC-RI-MALS in NaNO₃/NaN₃.
Abbreviations:
g = graft;
b = block;
stat = statistical.

### Minor Notes

Just as a note, and according to the art—the term “pharmaceutically acceptable” means that which is useful in preparing a pharmaceutical composition and is preferably non-toxic—i.e. a “pharmaceutically acceptable salt” is a preferably a non-toxic salt. Numerous different types of pharmaceutically acceptable salts are known to the skilled person.

### REFERENCE LIST

1: EP3331937B1

1. A graft copolymer, wherein the graft copolymer is a compound of formula Ia, a pharmaceutically acceptable salt thereof, or any stereoisomer or mixtures of stereoisomers, either of the compound of formula (Ia) or of any of its pharmaceutically acceptable salts, comprising homo-polypeptides or random or block or graft co-polypeptides: 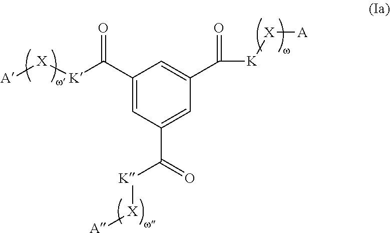
wherein A, A′ and A″ are each independently selected from a polymer comprising a backbone of repeating structural units of formula (I); and each of A, A′ and A″ sub-units may be same or different;
wherein K, K′ and K″ are each independently selected from —O—, —NH—;
X is selected from 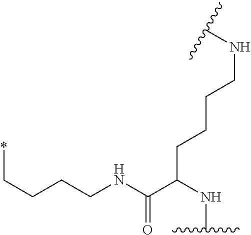 [SMILES: *CCCCNC(=O)C(CCCCN*)N*]
wherein ω, ω′ and ω″ are an integer from 0 to 1; each wavy lines denote the attaching points to A, A′ or A″; and “*” denotes the attaching point to K, K′ or K″;
and wherein formula (I) is a graft copolymer compound comprising a backbone of repeating structural units of formula (I) 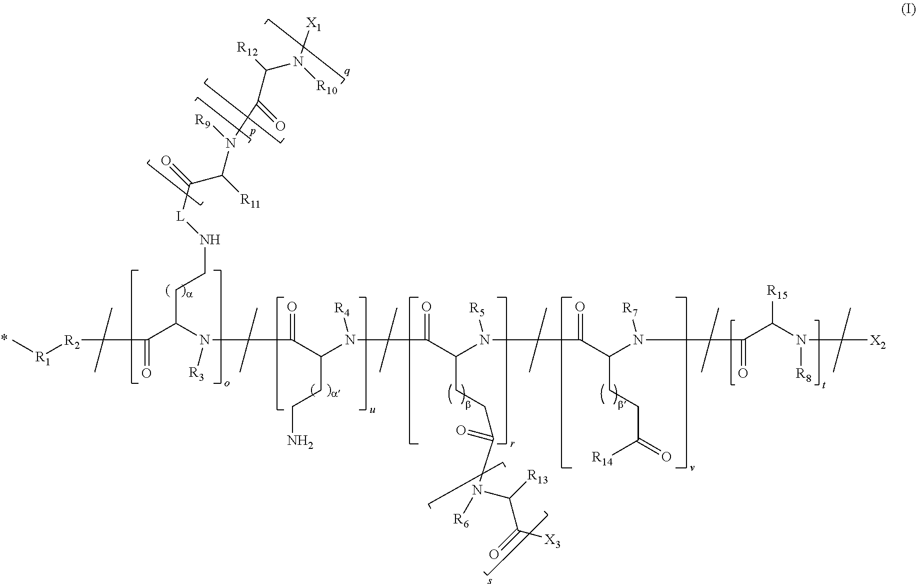
wherein though the main repeating units defined by square brackets with their numerical value o, r, t, u, and v, respectively, are shown in a particular order for convenience of description, the main repeating units may be present in any order and may be block or randomly present;
wherein each of the main repeating unit defined by square brackets with the numerical value “o” may be same or different between each other, may comprise blocks of secondary monomer units defined by square brackets with the numerical value “p” and “q” which may be same or different from each other according to the definitions of its different substituents;
wherein each of the main repeating units defined by square brackets with the numerical value “r” may be same or different between each other, may comprise blocks of secondary monomer units defined by square brackets with the numerical value “s” and “t” which may be same or different from each other according to the definitions of its different substituents;
wherein the “*” denotes an attaching point;
wherein “/” indicates that the sequential order of the repeating units on both sides of the sign are arbitrary;
R₁ is a biradical selected from the group consisting of 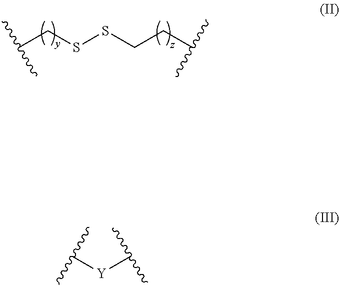
wherein the wavy lines denote the attaching points;
wherein y and z are integers independently ranging from 1 to 20; and
wherein Y is a biradical with a molecular weight (MW) from 5 to 3000 g/mol
R₂ is selected from —O—, —NH—;
wherein o and r are an integer each independently selected from 0 to 5000; with the proviso that at least one of o or r is ≠0;
p and s are an integer each independently selected from 1 to 1000;
q, t, u and v are an integer each independently selected from 0 to 1000;
α, α′ are each independently an integer selected from 1 to 4;
β, and β′ are each independently an integer selected from 0 to 3;
each R₃ is independently selected from H and —CH₃;
each R₄ is independently selected from H and —CH₃;
each R₅ is independently selected from H and —CH₃;
each R₆ is independently selected from H and —CH₃;
each R₇ is independently selected from H and —CH₃;
each R₈ is independently selected from H and —CH₃;
each R₉ is independently selected from H and —CH₃;
each R₁₀ is independently selected from H and —CH₃;
each R₁₅ is independently selected from H and —CH₃;
each R₁₁ and R₁₂ is independently selected from H, an imaging or labeling agent, a cell-targeting agent, and a radical of formula (XII), (XIII), (XIV), (XV), (XVI), (XVII), (XVIII), (XIX), (XX), (XXI), (XXII) or (XXIII) 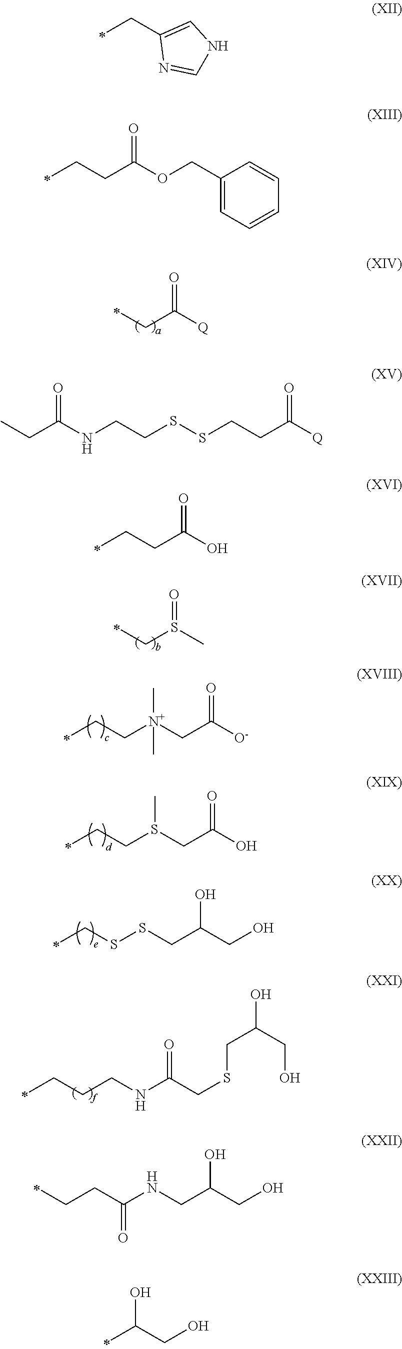
wherein Q is selected from —OH, an imaging or labeling agent, a cell-targeting agent, and a, b, c, d, e and f are integers each independently selected from 0 to 4;
each R₁₃ is independently selected from H, —CH₃, an imaging or labeling agent, a cell-targeting agent, and a radical of formula (XII), (XIII), (XIX), (XX), (XXI), (XXII), (XVIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), or (XXXVIII) 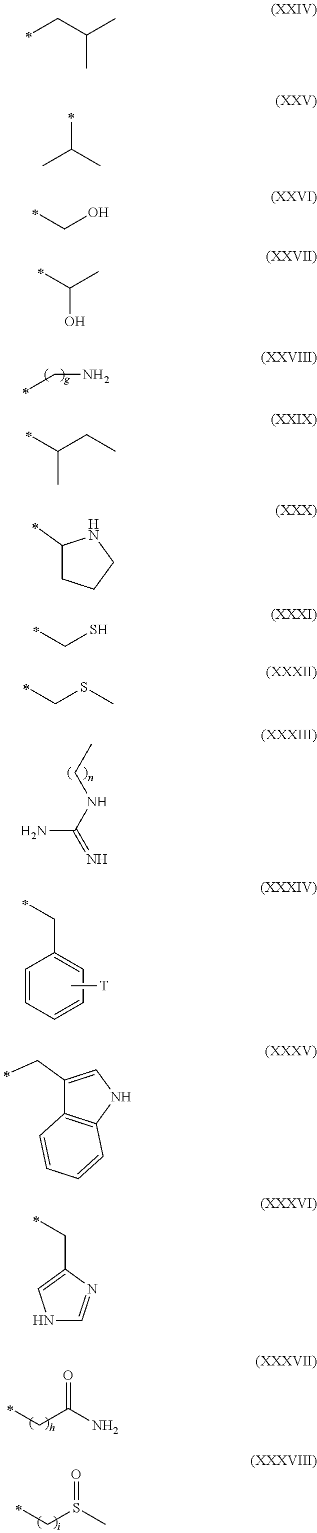
wherein g, h, i, and j are integers each independently selected from 0 to 4;
T is selected from H, and —OH;
each R₁₄ is independently selected from H, —OH, an imaging or labeling agent, and a cell-targeting agent;
wherein L is selected from a single bond, and a biradical of formula 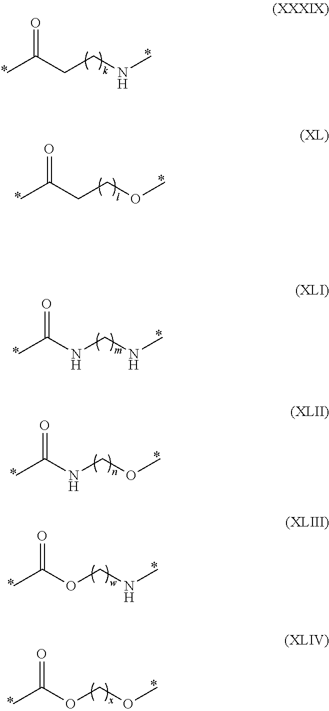
wherein k, l, m, n, w, and x are integers each independently selected from 0 to 4;
wherein the “*” denotes the attaching points;
each X₁ is independently selected from H, —C(O)H, —S(O)H, —NHCH(CH₃)₂, —(C₁-C₄)alkylNH₂, —(C₁-C₄)alkylNHCH₃, —(C₁-C₄)alkylN(CH₃)₂, —O—(C₁-C₄)alkyl-NH₂, —O—(C₁-C₄)alkyl-NHCH₃, —O—(C₁-C₄)alkyl-N(CH₃)₂, an imaging or labeling agent, a cell-targeting agent, and a radical of formula (XVI), (XVII), (XVIII), (XIX), (XX), (XXI), (XXII) or (XXIII);
each X₂ is independently selected from H, —C(O)H, —S(O)H, —NHCH(CH₃)₂, —(C₁-C₄)alkylNH₂, —(C₁-C₄)alkylNHCH₃, —(C₁-C₄)alkylN(CH₃)₂, —O—(C₁-C₄)alkyl-NH₂, —O—(C₁-C₄)alkyl-NHCH₃, —O—(C₁-C₄)alkyl-N(CH₃)₂, an imaging or labeling agent, a cell-targeting agent, and a radical of formula (XVI), (XVII), (XVIII), (XIX), (XX), (XXI), (XXII) or (XXIII); and
each X₃ is independently selected from H, —C(O)H, —S(O)H, —NHCH(CH₃)₂, —(C₁-C₄)alkylNH₂, —(C₁-C₄)alkylNHCH₃, —(C₁-C₄)alkylN(CH₃)₂, —O—(C₁-C₄)alkyl-NH₂, —O—(C₁-C₄)alkyl-NHCH₃, —O—(C₁-C₄)alkyl-N(CH₃)₂, an imaging or labeling agent, a cell-targeting agent, and a radical of formula (XVI), (XVII), (XVIII), (XIX), (XX), (XXI), (XXII) or (XXIII).

2. The graft copolymer of claim 1, wherein the Y biradical of R₁ is a biradical selected from the group consisting of —NH—, —NH(C₁-C₆)alkyl-, —O—, —(C₁-C₆)alkyl-COO—, a straight or branched —(C₁-C₃₀)alkylene-, and a biradical of formula (IV), (V), (VI), (VII), (VIII), (IX), (X), or (XI) 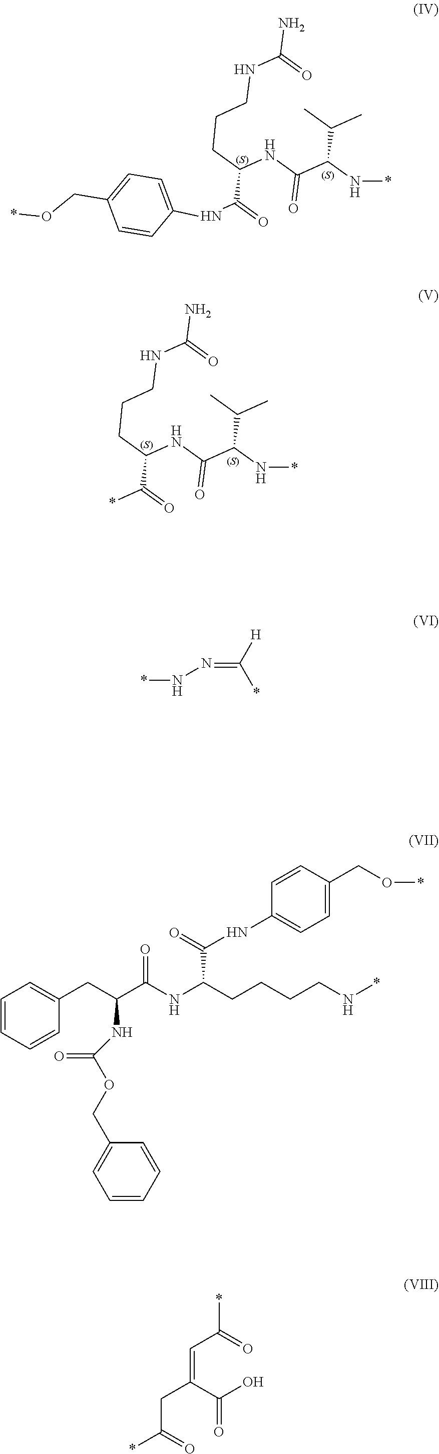 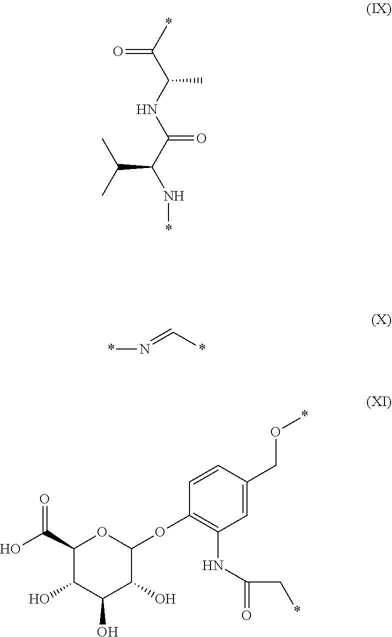
wherein the “*” denotes the attaching points;
wherein the —(C₁-C₃₀)alkylene biradical of Y is optionally substituted with one or more radical selected from the group consisting of —OH, —NRₐR_{b}, —SH₂, —NHNH₂, —COOR_{c}, —CF₃, —OCF₃, and halogen, wherein Rₐ, R_{b} and R_{c} are radicals independently selected from the group consisting of H, -phenyl, —(C₁-C₃₀)alkyl, —(C₂-C₃₀)alkenyl, —(C₁-C₃₀)alkylphenyl, and -phenyl(C₁-C₃₀)alkyl.

3. The graft copolymer of claim 1, wherein the molecular weight (MW) is from 3 to 500000 kilodaltons (kDa) and wherein the graft copolymer is a unimolecular structure with a radius from 4-300 nm.

4. The graft copolymer of claim 1, wherein ω, ω′ and ω″ integer is 0, R₁ is ethylene and R₂=—NH—.

5. The graft copolymer of claim 1, wherein the integer “o” is ≠0; wherein L is a single bond; wherein α is an integer of 3; and wherein the integers “r” and “v” are 0.

6. The graft copolymer of claim 5, wherein the value of integer “u” is less than 25% of the numerical value of integer “o”.

7. The graft copolymer of claim 5, wherein the numerical value of integer “o” is from 5 to 3000 and wherein the numerical value of integer “p” is from 3 to 1000.

8. The graft copolymer of claim 5, wherein each R₁₁ and R₁₂ is independently selected from H and a radical of formula (XII), (XIII), (XIV) and (XVI).

9. The graft copolymer of claim 5, wherein the graft copolymer is a compound comprising a compound selected from the group consisting of:
St-PLys-g-PGlu
St-PLys-g-PHis
St-PLys-g-PHis-b-PGlu
St-PLys-g-PHis-b-PSar
St-PLys-g-PBG
St-PLys-g-PBG-b-PGlu
St-PLys-g-PBG-b-PSar
St-PLys-g-PSar
St-PLys-g-PSar-g-VitE; and
St-(PLys-g-PGlu)-stat-PSar, and
wherein the term “St” is the star structure of formula (Ia) of claim 1; and
wherein the term “PBG” is poly-Benzyl glutamate.

10. The graft copolymer of claim 1, wherein the integer “r” is ≠0; wherein β is an integer of 1; and the integers “o” and “u” are 0.

11. The graft copolymer of claim 10, wherein the numerical value of integer “r” is from 3 to 1000; and wherein the numerical value of integer “s” is from 2 to 400.

12. The graft copolymer of claim 10, wherein R₁₃ is independently selected from H and a radical of formula (XII), (XIII), (XXI) and (XXVIII).

13. The graft copolymer of claim 10, wherein the graft copolymer is a compound comprising a compound selected from the group consisting of:
St-PGlu-g-PSar
St-PGlu-g-PHis
St-PGlu-g-PLys
St-PGlu-g-PLysTG; and
St-PGlu-g-PSar-g-PHis; and
wherein the term “St” is the star structure of formula (Ia) of claim 1; and
wherein PLysTG is poly-lysine-thioglycerated.

14. A polymer complex or a pharmaceutical composition comprising a graft copolymer of claim 1, and at least a pharmaceutical, veterinary or cosmetical active agent.

15. A method for delivering a pharmaceutical active agent into a target cell, which comprises administering a composition that contains the polymer complex of claim 14 to an animal, including human, so that the complex is getting into physical contact with the target cell and thereby delivers the pharmaceutical active agent into the cell.

16. A process for preparing a compound which is structurally different from a graft copolymer starting compound of claim 1, comprising following steps:
(i) using a compound of claim 1 as a starting compound; and
(ii) making structural changes to the compound of step (i) to obtain a compound, which is structurally different from the starting compound of (i);
and wherein the obtained structurally different compound of step (ii) is a compound, which is capable of forming a polymer complex comprising the obtained structurally different compound of step (ii), and at least one pharmaceutical, veterinary or cosmetical active agent, and wherein the active agent is an active agent that the starting compound of step (i) is also capable of forming a polymer complex with.
